SEQUENCE LISTING (106 sequences)

SEQ ID NO: 1 - DNA, 26 bp, Artificial Sequence
  PCR primer
cccacttcaccacactctactcagtc

SEQ ID NO: 2 - DNA, 26 bp, Artificial Sequence
  PCR primer
gctggacgcagtgtcttctcccttcc

SEQ ID NO: 3 - DNA, 21 bp, Artificial Sequence
  siRNA sequence
uuuacaauuuggaaguagcug

SEQ ID NO: 4 - DNA, 21 bp, Artificial Sequence
  siRNA sequence
aaaugugcaauagccauccgu

SEQ ID NO: 5 - DNA, 21 bp, Artificial Sequence
  si RNA
ucuuucaaagguaugccaagu

SEQ ID NO: 6 - DNA, 21 bp, Artificial Sequence
  si RNA
uaugugacaagugaugucuga

SEQ ID NO: 7 - DNA, 21 bp, Artificial Sequence
  si RNA
uguauuaaccucuucacuggg

SEQ ID NO: 8 - DNA, 21 bp, Artificial Sequence
  si RNA
uaagauuggagagaaauucca

SEQ ID NO: 9 - DNA, 21 bp, Artificial Sequence
  si RNA
uucaagucggaacucgugcag

SEQ ID NO: 10 - DNA, 21 bp, Artificial Sequence
  si RNA
uuguuuuggcaguaaauugug

SEQ ID NO: 11 - DNA, 21 bp, Artificial Sequence
  si RNA
uuacuuugugguucucagggu

SEQ ID NO: 12 - DNA, 21 bp, Artificial Sequence
  si RNA
uuauauuccucuaugaagucu

SEQ ID NO: 13 - DNA, 21 bp, Artificial Sequence
  si RNA
uagccugccucugucaccuga

SEQ ID NO: 14 - DNA, 21 bp, Artificial Sequence
  si RNA
uuauagagcacaaucugucau

SEQ ID NO: 15 - DNA, 21 bp, Artificial Sequence
  si RNA
uuccacaacagaaugacggca

SEQ ID NO: 16 - DNA, 21 bp, Artificial Sequence
  si RNA
uaaucuuauaguaaucaggga

SEQ ID NO: 17 - DNA, 21 bp, Artificial Sequence
  si RNA
gaaggauacacguuuggauga

SEQ ID NO: 18 - DNA, 21 bp, Artificial Sequence
  si RNA
uaagucuggagaaguaucgcu

SEQ ID NO: 19 - DNA, 21 bp, Artificial Sequence
  si RNA
uuucuggagaacacauaagug

SEQ ID NO: 20 - DNA, 21 bp, Artificial Sequence
  si RNA
uuaauauuucuucaacagcua

SEQ ID NO: 21 - DNA, 21 bp, Artificial Sequence
  si RNA
uucuaguuugacauucuucag

SEQ ID NO: 22 - DNA, 21 bp, Artificial Sequence
  si RNA
uauaacacacuguacacagug

SEQ ID NO: 23 - DNA, 21 bp, Artificial Sequence
  siRNA
uucuugguucagcugaucgua

SEQ ID NO: 24 - DNA, 21 bp, Artificial Sequence
  si RNA molecule
uugaggugguaacugcuccga

SEQ ID NO: 25 - DNA, 21 bp, Artificial Sequence
  si RNA molecule
uuauagucuucguaccacuug

SEQ ID NO: 26 - DNA, 21 bp, Artificial Sequence
  si RNA molecule
uuuuguucucuucaacacugg

SEQ ID NO: 27 - DNA, 21 bp, Artificial Sequence
  si RNA molecule
uuaauuagagcauuuguagau

SEQ ID NO: 28 - DNA, 21 bp, Artificial Sequence
  si RNA molecule
uuuucugcuacaugcuucggu

SEQ ID NO: 29 - DNA, 21 bp, Artificial Sequence
  si RNA molecule
uguagucgcucaucaagguug

SEQ ID NO: 30 - DNA, 21 bp, Artificial Sequence
  si RNA molecule
uuuuucugaaguguggcaugu

SEQ ID NO: 31 - DNA, 21 bp, Artificial Sequence
  si RNA molecule
uacuauugcagaaacacgcuu

SEQ ID NO: 32 - DNA, 21 bp, Artificial Sequence
  si RNA molecule
uuagaaguucuuguauuccua

SEQ ID NO: 33 - DNA, 21 bp, Artificial Sequence
  si RNA molecule
uaugauuucaaauucacccca

SEQ ID NO: 34 - DNA, 21 bp, Artificial Sequence
  si RNA molecule
uacguucauauuuauaaccuu

SEQ ID NO: 35 - DNA, 21 bp, Artificial Sequence
  si RNA molecule
uuaauguacacauagaucuua

SEQ ID NO: 36 - DNA, 21 bp, Artificial Sequence
  si RNA molecule
uucaaguuuggcaucaaugau

SEQ ID NO: 37 - DNA, 21 bp, Artificial Sequence
  si RNA molecule
uacuccugucuugucgcagug

SEQ ID NO: 38 - DNA, 21 bp, Artificial Sequence
  si RNA molecule
uagguuuccucuuccuagagu

SEQ ID NO: 39 - DNA, 21 bp, Artificial Sequence
  si RNA molecule
uuguuugauaaggugauccuu

SEQ ID NO: 40 - DNA, 21 bp, Artificial Sequence
  si RNA molecule
uuuuggaagagcuacuuccug

SEQ ID NO: 41 - DNA, 21 bp, Artificial Sequence
  si RNA molecule
uacaacacggauucugucuca

SEQ ID NO: 42 - DNA, 21 bp, Artificial Sequence
  si RNA molecule
uagaaugacggcaucaucucc

SEQ ID NO: 43 - DNA, 21 bp, Artificial Sequence
  si RNA molecule
uauuuuaauaaggugaugcug

SEQ ID NO: 44 - DNA, 21 bp, Artificial Sequence
  si RNA molecule
uauauguaugacuaaaggcuu

SEQ ID NO: 45 - DNA, 21 bp, Artificial Sequence
  si RNA molecule
uuauuaaauagguaaaggguu

SEQ ID NO: 46 - DNA, 21 bp, Artificial Sequence
  si RNA molecule
auuagcaagugaacguuuuga

SEQ ID NO: 47 - DNA, 21 bp, Artificial Sequence
  si RNA molecule
ucgauaaugaccacacucggu

SEQ ID NO: 48 - DNA, 21 bp, Artificial Sequence
  si RNA molecule
uugcauuuucacauuuuccgu

SEQ ID NO: 49 - DNA, 21 bp, Artificial Sequence
  si RNA molecule
uuauuuacaaaucuauaacuu

SEQ ID NO: 50 - DNA, 21 bp, Artificial Sequence
  si RNA molecule
uuuguucgccacuugcucuuc

SEQ ID NO: 51 - DNA, 21 bp, Artificial Sequence
  si RNA molecule
uuaguuuacaacuauucugua

SEQ ID NO: 52 - DNA, 21 bp, Artificial Sequence
  si RNA molecule
uuuguugauaucuagacuuag

SEQ ID NO: 53 - DNA, 21 bp, Artificial Sequence
  si RNA molecule
uuugcuuauugacacuaccga

SEQ ID NO: 54 - DNA, 21 bp, Artificial Sequence
  si RNA molecule
uugagugcuagacucaugguu

SEQ ID NO: 55 - DNA, 21 bp, Artificial Sequence
  si RNA molecule
auugucauugaugucaucguu

SEQ ID NO: 56 - DNA, 21 bp, Artificial Sequence
  si RNA molecule
uucgacuguuuugucuucguu

SEQ ID NO: 57 - DNA, 21 bp, Artificial Sequence
  si RNA molecule
uucauacagucugugucaucc

SEQ ID NO: 58 - DNA, 21 bp, Artificial Sequence
  si RNA molecule
uuauauugaauguuucuugug

SEQ ID NO: 59 - DNA, 21 bp, Artificial Sequence
  si RNA molecule
uuuaaauuuaucauucuugga

SEQ ID NO: 60 - DNA, 21 bp, Artificial Sequence
  si RNA molecule
uuaauugagaacuauagcccu

SEQ ID NO: 61 - DNA, 21 bp, Artificial Sequence
  si RNA molecule
uuagaguccuguaugauagga

SEQ ID NO: 62 - DNA, 21 bp, Artificial Sequence
  si RNA molecule
uuuaacuuguaguacaaugga

SEQ ID NO: 63 - DNA, 21 bp, Artificial Sequence
  si RNA molecule
uaaaugcucaaauauuacuau

SEQ ID NO: 64 - DNA, 21 bp, Artificial Sequence
  si RNA molecule
uucaaaggagaacucauuuuc

SEQ ID NO: 65 - DNA, 21 bp, Artificial Sequence
  si RNA molecule
uaaagucucagucuccucuug

SEQ ID NO: 66 - DNA, 21 bp, Artificial Sequence
  si RNA molecule
uuauauuguacuuugacaggg

SEQ ID NO: 67 - DNA, 21 bp, Artificial Sequence
  si RNA molecule
uuaggaaagagaguguguagu

SEQ ID NO: 68 - DNA, 21 bp, Artificial Sequence
  si RNA molecule
uauauucuucagaaaauucua

SEQ ID NO: 69 - DNA, 21 bp, Artificial Sequence
  si RNA molecule
uauauuaaccauauacaugug

SEQ ID NO: 70 - DNA, 21 bp, Artificial Sequence
  si RNA molecule
uuacacaucacuucacaacug

SEQ ID NO: 71 - DNA, 21 bp, Artificial Sequence
  si RNA molecule
uauuaauuucaucuuuuucuu

SEQ ID NO: 72 - DNA, 21 bp, Artificial Sequence
  si RNA molecule
uccgcuuuaggaaagagagug

SEQ ID NO: 73 - DNA, 21 bp, Artificial Sequence
  si RNA molecule
uuaaaauagauuuguauccua

SEQ ID NO: 74 - DNA, 21 bp, Artificial Sequence
  si RNA molecule
uucuguauuaauaacaagcuu

SEQ ID NO: 75 - DNA, 21 bp, Artificial Sequence
  si RNA molecule
uucauugcaauaauacuuguu

SEQ ID NO: 76 - DNA, 21 bp, Artificial Sequence
  si RNA molecule
uaacuuauagggauauccuga

SEQ ID NO: 77 - DNA, 21 bp, Artificial Sequence
  si RNA molecule
uuuaauagcauucuuaucaua

SEQ ID NO: 78 - DNA, 21 bp, Artificial Sequence
  si RNA molecule
uauguuuauucuuuaguauuc

SEQ ID NO: 79 - DNA, 21 bp, Artificial Sequence
  si RNA molecule
uuaauguucuugauucucuug

SEQ ID NO: 80 - DNA, 21 bp, Artificial Sequence
  si RNA molecule
uuaauuacugcaaacaaagaa

SEQ ID NO: 81 - DNA, 21 bp, Artificial Sequence
  si RNA molecule
uuucuguuucuauuaguucuu

SEQ ID NO: 82 - DNA, 21 bp, Artificial Sequence
  si RNA molecule
uuuaccuuuaugauucaucug

SEQ ID NO: 83 - DNA, 21 bp, Artificial Sequence
  si RNA molecule
uugauaaucagcugaacucug

SEQ ID NO: 84 - DNA, 21 bp, Artificial Sequence
  si RNA molecule
uuaauauuuacauucaagcag

SEQ ID NO: 85 - DNA, 21 bp, Artificial Sequence
  si RNA molecule
uuaacaucuguguaaacucug

SEQ ID NO: 86 - DNA, 21 bp, Artificial Sequence
  si RNA molecule
uauaucuaucaucucucucug

SEQ ID NO: 87 - DNA, 21 bp, Artificial Sequence
  si RNA molecule
uuuauuucuauuaauaagcug

SEQ ID NO: 88 - DNA, 21 bp, Artificial Sequence
  si RNA molecule
uuucuuuaucuucugcaucuu

SEQ ID NO: 89 - DNA, 21 bp, Artificial Sequence
  si RNA molecule
uuguaaaucaaaguauaccuu

SEQ ID NO: 90 - DNA, 21 bp, Artificial Sequence
  si RNA molecule
uucguaaauaucaucuuagau

SEQ ID NO: 91 - DNA, 21 bp, Artificial Sequence
  si RNA molecule
uuucaguuugagaacagccuu

SEQ ID NO: 92 - DNA, 21 bp, Artificial Sequence
  si RNA molecule
uuucucaugggucugauccgg

SEQ ID NO: 93 - DNA, 21 bp, Artificial Sequence
  si RNA molecule
uaguguaauacaaugcuccgu

SEQ ID NO: 94 - DNA, 21 bp, Artificial Sequence
  si RNA molecule
uauagucauauaaaucuucug

SEQ ID NO: 95 - DNA, 21 bp, Artificial Sequence
  si RNA molecule
uuucuuuauagcucuagggua

SEQ ID NO: 96 - DNA, 21 bp, Artificial Sequence
  si RNA molecule
uuaucuugcuguucaucaccu

SEQ ID NO: 97 - DNA, 21 bp, Artificial Sequence
  si RNA molecule
uuaucuuuucuauaaucccga

SEQ ID NO: 98 - DNA, 21 bp, Artificial Sequence
  si RNA molecule
uaaugucucagaaguaaagug

SEQ ID NO: 99 - DNA, 21 bp, Artificial Sequence
  si RNA molecule
uuuauauacacacaaacugua

SEQ ID NO: 100 - DNA, 21 bp, Artificial Sequence
  si RNA molecule
uugcucuuuaucuccuaucaa

SEQ ID NO: 101 - DNA, 21 bp, Artificial Sequence
  si RNA molecule
uacauugauaaccaaaaggug

SEQ ID NO: 102 - DNA, 21 bp, Artificial Sequence
  si RNA molecule
uuuacuuucaucuagcuucag

SEQ ID NO: 103 - DNA, 21 bp, Artificial Sequence
  siRNA
cacaatttactgccaaaacaa

SEQ ID NO: 104 - DNA, 21 bp, Artificial Sequence
  siRNA
cagctacttccaaattgtaaa

SEQ ID NO: 105 - DNA, 21 bp, Artificial Sequence
  siRNA
tggaatttctctccaatctta

SEQ ID NO: 106 - DNA, 21 bp, Artificial Sequence
  siRNA
aggaattataatgcttatcta

(19) United States
(12) Patent Application Publication
(10) Pub. No.: US 2017/0211048 A1
(43) Pub. Date: Jul. 27, 2017

(54) ENHANCED REPROGRAMMING TO IPS CELLS

(71) Applicants: Boehringer Ingelheim International GmbH, Ingelheim am Rhein (DE); IMBA - INSTITUT FUER MOLEKULARE BIOTECHNOLOGIE GMBH, Vienna (AT)

(72) Inventors: Ulrich ELLING, Vienna (AT); Barbara HOPFGARTNER, Perchtoldsdorf (AT); Josef PENNINGER, Vienna (AT); Johannes ZUBER, Vienna (AT)

(21) Appl. No.: 15/328,122

(22) PCT Filed: Jul. 23, 2015
(86) PCT No.: PCT/EP2015/066887

§ 371 Date: Jan. 23, 2017

(30) Foreign Application Priority Data
Jul. 25, 2014 (EP) 14178552.7

(51) Int. Cl. C12N 5/074; C12N 15/113

(52) U.S. Cl. CPC C12N 5/0696; C12N 15/113; C12N 2506/1307; C12N 2310/14; C12N 2310/531; C12N 2501/40; C12N 2501/606; C12N 2501/70

(57) ABSTRACT

The present invention concerns a method of preparing a population of iPS cells comprising reducing the amount and/or activity of one or more components of the CAF1 complex, and/or one or more components of the SUMO pathway, in a population of target cells, and (ii) optionally isolating the iPS cells from the target cell population.

### BACKGROUND OF THE INVENTION

The present invention relates to a method for improving the efficiency of iPS formation.

### BACKGROUND

Since its discovery, cellular reprogramming to pluripotency has become a broadly used experimental tool. Beyond its great utility in basic and biomedical research, iPS reprogramming is believed to be applicable for a wide range of medical applications such as the generation of patient-specific tissue for cellular therapy. However, the process of iPS reprogramming remains very inefficient and stochastic in nature, which diminishes its utility for many applications, particularly if the source of somatic cells is limited. While the major roadblock preventing efficient iPS reprogramming is thought to lie in the hard-wired epigenetic landscape, the key mechanisms and factors contributing to this roadblock remain incompletely understood.

There exists a need in the art for improved methods for reprogramming mammalian cells.

### SUMMARY OF THE INVENTION

Accordingly, a first aspect of the invention provides a method of preparing a population of iPS cells comprising reducing the amount and/or activity of one or more components of the CAF1 complex, and/or one or more components of the SUMO pathway, in a population of target cells, and (ii) optionally isolating the iPS cells from the target cell population.

The present inventors performed a functional genetic screen to systematically identify chromatin-associated factors involved in preventing iPS reprogramming. From this screen they observed dramatic increase in cell reprogramming efficacy when certain genetic factors including components of the CAF1 complex, or SUMO pathway, are reduced. The degree of these effects exceeds by far the increase observed for previously established factors and provides an important advance in the development of therapies using cellular reprogramming.

Strategies to suppressing these genetic factors by using agents such as RNAi, other genetic techniques or small-molecule inhibitors, provide novel powerful tools with a wide spectrum of research and biomedical applications. For example:
Efficient generation of iPS cells for medical applications from material of limited quality and/or quantity: The method of the invention provides for efficient and more rapid generation of patient specific iPS cells for the study of disease as well as for regeneration of tissue in vitro. This will enable iPS generation from less material harvested in biopsies.
Direct tissue reprogramming: Several of the identified genetic factors (e.g. the CAF1 complex components CHAF1A/B, and SETDB1) are known to have ubiquitous functions in preserving epigenetic states throughout cell division. Hence it is likely that their inhibition can erase epigenetic memory and thereby enhance cellular reprogramming in many tissue contexts. Beyond enhancing iPS regimens, the inhibition of these factors may also facilitate reprogramming between other cellular contexts (e.g. direct reprogramming of fibroblasts into neurons).
Regenerative medicine in situ: The data provided herein suggest that the suppression of the identified factors, in particular CHAF1A/B and UBE2I, strongly facilitates cell fate switches by overwriting epigenetic memory. Therefore, strategies aimed at inhibiting these factors and their associated complexes and pathways, particularly the CAF1 complex and SUMOylation, can be used to improve regenerative processes involving de-differentiation in vivo such as repair mechanisms following tissue injury (e.g. stroke, or heart attack, bone marrow reconstitution, etc.).
Development of simpler and safer iPS reprogramming protocols: The removal of epigenetic roadblocks through inhibition of the identified genetic factors (alone or in combination) can be used to enable the development of new effective reprogramming protocols that are based on fewer ectopically expressed reprogramming factors and/or require less time. Particularly desirable for biomedical applications would be the development of iPS regimens that do neither require c-MYC nor viral gene delivery, since both these factors pose substantial biosafety risks. More generally, unlike existing reprogramming factors, the identified genetic factors represent a first set of targets for chemically induced reprogramming.
The study of reprogramming: A better mechanistic understanding of the iPS process has critical relevance for the further optimization and biomedical application of reprogramming technology. However, the fact that existing protocols for reprogramming remain highly inefficient and stochastic in nature complicates the dynamic study of this process. Approaches to massively improve reprogramming efficiency (as provided by the method of the present invention) will provide a better handle on cells in transition from differentiated to iPS state.
Efficient generation of iPS cells for research even from material of limited quality and/or quantity: As the process of reprogramming is inefficient, in particular with terminally differentiated, aged or poorly proliferative cell material, it is important to optimize reprogramming in order to be able to derive e.g. patient specific iPS cells for the study of underlying disease mechanisms. An optimized iPS protocol will facilitate iPS generation from cell types or organisms currently refractory to iPS formation.

### DETAILED DESCRIPTION OF THE INVENTION

Accordingly, a first aspect of the invention provides a method of preparing a population of iPS cells comprising reducing the amount and/or activity of one or more components of the CAF1 complex, and/or one or more components of the SUMO pathway in a population of target cells, and (ii) optionally isolating the iPS cells from the target cell population.

From a functional genetic screen to systematically identify chromatin-associated factors involved in preventing iPS reprogramming, the inventors observed dramatic increase in cell reprogramming efficacy when certain genetic factors including components of the CAF1 complex, or SUMO pathway, are reduced. The method of the invention allows for an increase of reprogramming efficiency of several orders of magnitude and generated iPSCs two or three times faster compared to controls.

Until the present invention, it had not previously been known or even suspected that modulating one or more components of the CAF1 complex, and/or one or more SUMO pathway in a population of target cells would lead to such a dramatic increase in cell reprogramming efficacy.

Chromatin assembly factor 1 (CAF-1) is a nuclear complex that functions in de novo assembly of nucleosomes during DNA replication and nucleotide excision repair. Nucleosome assembly is a two-step process, involving initial deposition of a histone H3/H4 tetramer onto DNA, followed by the deposition of a pair of histone H2A/H2B dimers. CAF-1 interacts with PCNA and localizes to DNA replication and DNA repair foci, where it functions to assemble newly synthesized histone H3/H4 tetramers onto replicating DNA. Assembly of histone H2A/H2B dimers requires additional assembly factors. The CAF-1 complex consists of three proteins: CHAF1A (p150), CHAF1B (p60) and RBAP48 (p48 or RBBP4). CHAF1A and CHAF1B proteins are specific for the CAF-1 complex, while RBAP48 is a component of multiple chromatin modifying complexes.

By “components of the CAF1 complex”, the present invention includes in the claimed method a step of reducing the amount and/or activity of one or more of the components of the CAF1 complex provided herein, i.e. CHAF1A, CHAF1B and RBBP4.

In a preferred embodiment of the invention, the step of reducing the amount and/or activity of the CAF1 complex comprises reducing the amount and/or activity of CHAF1A, CHAF1B and/or RBBP4 protein in the target cells.

CHAF1A, CHAF1B and RBBP4 are known in the art and information concerning their amino acid sequence and the nucleic acid sequence of the associated genes can be readily identified by the skilled person.

By way of example, human CHAF1A is listed in the NCBI database as Gene ID: 10036. The entry for Chaf1a includes information including amino acid and nucleic acid sequences. http://www.ncbi.nlm.nih.gov/gene/10036

Human CHAF1B is listed in the NCBI database as Gene ID: 8208. The entry for Chaf1b includes information including amino acid and nucleic acid sequences. http://www.ncbi.nlm.nih.gov/gene/8208

Human RBBP4 is listed in the NCBI database as Gene ID: 5928. The entry for Chaf1a includes information including amino acid and nucleic acid sequences. http://www.ncbi.nlm.nih.gov/gene/5928

The SUMO pathway modifies hundreds of proteins that participate in diverse cellular processes. The SUMO pathway is well known in the art. The CAF-1 complex consists of SUMO1, SUMO2, SUMO3, SUMO4, SAE1, UBA2, UBE2I, PIAS1, PIAS2, PIAS3, PIAS4, RANBP2, CBX4, NSMCE2, MUL1, HDAC4, HDAC7, TOPORS, FUS, RASD2, TRAF7, SENP1, SENP2, SENP3, SENP5, SENP6 or SENP7.

By “components of the SUMO pathway”, the present invention includes in the claimed method a step of reducing the amount and/or activity of one or more of the components of the SUMO pathway provided herein, i.e. SUMO1, SUMO2, SUMO3, SUMO4, SAE1, UBA2, UBE2I, PIAS1, PIAS2, PIAS3, PIAS4, RANBP2, CBX4, NSMCE2, MUL1, HDAC4, HDAC7, TOPORS, FUS, RASD2, TRAF7, SENP1, SENP2, SENP3, SENP5, SENP6 or SENP7.

In a preferred embodiment of the invention, step of reducing the amount and/or activity of one or more components of the SUMO pathway comprises reducing the amount and/or activity of SUMO1, SUMO2, SUMO3, SUMO4, SAE1, UBA2, UBE2I, PIAS1, PIAS2, PIAS3, PIAS4, RANBP2, CBX4, NSMCE2, MUL1, HDAC4, HDAC7, TOPORS, FUS, RASD2, TRAF7, SENP1, SENP2, SENP3, SENP5, SENP6 or SENP7.

SUMO1, SUMO2, SUMO3, SUMO4, SAE1, UBA2, UBE2I, PIAS1, PIAS2, PIAS3, PIAS4, RANBP2, CBX4, NSMCE2, MUL1, HDAC4, HDAC7, TOPORS, FUS, RASD2, TRAF7, SENP1, SENP2, SENP3, SENP5, SENP6 and SENP7 are known in the art and information concerning their amino acid sequence and the nucleic acid sequence of the associated genes can be readily identified by the skilled person.

By way of example, human SUMO1 is listed in the NCBI database as Gene ID: 7341. The entry for SUMO1 includes information including amino acid and nucleic acid sequences. http://www.ncbi.nlm.nih.gov/gene/7341

Human SUMO2 is listed in the NCBI database as Gene ID: 6613. The entry for SUMO2 includes information including amino acid and nucleic acid sequences. http://www.ncbi.nlm.nih.gov/gene/6613

Human SUMO3 is listed in the NCBI database as Gene ID: 6612. The entry for SUMO3 includes information including amino acid and nucleic acid sequences. http://www.ncbi.nlm.nih.gov/gene/6612

Human SUMO4 is listed in the NCBI database as Gene ID: 387082. The entry for SUMO4 includes information including amino acid and nucleic acid sequences. http://www.ncbi.nlm.nih.gov/gene/387082

Human SAE1 is listed in the NCBI database as Gene ID: 10055. The entry for SAE1 includes information including amino acid and nucleic acid sequences. http://www.ncbi.nlm.nih.gov/gene/10055

Human UBA2 is listed in the NCBI database as Gene ID: 10054. The entry for UBA2 includes information including amino acid and nucleic acid sequences. http://www.ncbi.nlm.nih.gov/gene/10054

Human UBE2I is listed in the NCBI database as Gene ID: 7329. The entry for UBE2I includes information including amino acid and nucleic acid sequences. http://www.ncbi.nlm.nih.gov/gene/7329

Human PIAS1 is listed in the NCBI database as Gene ID: 8554. The entry for PIAS1 includes information including amino acid and nucleic acid sequences. http://www.ncbi.nlm.nih.gov/gene/8554

Human PIAS2 is listed in the NCBI database as Gene ID: 9063. The entry for PIAS2 includes information including amino acid and nucleic acid sequences. http://www.ncbi.nlm.nih.gov/gene/9063

Human PIAS3 is listed in the NCBI database as Gene ID: 10401. The entry for PIAS3 includes information including amino acid and nucleic acid sequences. http://www.ncbi.nlm.nih.gov/gene/10401

Human PIAS4 is listed in the NCBI database as Gene ID: 51588. The entry for PIAS4 includes information including amino acid and nucleic acid sequences. http://www.ncbi.nlm.nih.gov/gene/51588

Human RANBP2 is listed in the NCBI database as Gene ID: 5903. The entry for RANBP2 includes information including amino acid and nucleic acid sequences. http://www.ncbi.nlm.nih.gov/gene/5903

Human CBX4 is listed in the NCBI database as Gene ID: 8535. The entry for CBX4 includes information including amino acid and nucleic acid sequences. http://www.ncbi.nlm.nih.gov/gene/8535

Human NSMCE2 is listed in the NCBI database as Gene ID: 286053. The entry for NSMCE2 includes information including amino acid and nucleic acid sequences. http://www.ncbi.nlm.nih.gov/gene/286053

Human MUL1 is listed in the NCBI database as Gene ID: 79594. The entry for MUL1 includes information including amino acid and nucleic acid sequences. http://www.ncbi.nlm.nih.gov/gene/79594

Human HDAC4 is listed in the NCBI database as Gene ID: 9759. The entry for HDAC4 includes information including amino acid and nucleic acid sequences. http://www.ncbi.nlm.nih.gov/gene/9759

Human HDAC7 is listed in the NCBI database as Gene ID: 51564. The entry for HDAC7 includes information including amino acid and nucleic acid sequences. http://www.ncbi.nlm.nih.gov/gene/51564

Human TOPORS is listed in the NCBI database as Gene ID: 10210. The entry for TOPORS includes information including amino acid and nucleic acid sequences. http://www.ncbi.nlm.nih.gov/gene/10210

Human FUS is listed in the NCBI database as Gene ID: 2521. The entry for FUS includes information including amino acid and nucleic acid sequences. http://www.ncbi.nlm.nih.gov/gene/10210

Human RASD2 is listed in the NCBI database as Gene ID: 23551. The entry for RASD2 includes information including amino acid and nucleic acid sequences. http://www.ncbi.nlm.nih.gov/gene/23551

Human TRAF7 is listed in the NCBI database as Gene ID: 84231. The entry for TRAF7 includes information including amino acid and nucleic acid sequences. http://www.ncbi.nlm.nih.gov/gene/84231

Human SENP1 is listed in the NCBI database as Gene ID: 29843. The entry for SENP1 includes information including amino acid and nucleic acid sequences. http://www.ncbi.nlm.nih.gov/gene/29843

Human SENP2 is listed in the NCBI database as Gene ID: 59343. The entry for SENP2 includes information including amino acid and nucleic acid sequences. http://www.ncbi.nlm.nih.gov/gene/59343

Human SENP3 is listed in the NCBI database as Gene ID: 26168. The entry for SENP3 includes information including amino acid and nucleic acid sequences. http://www.ncbi.nlm.nih.gov/gene/26168

Human SENP5 is listed in the NCBI database as Gene ID: 205564. The entry for SENP5 includes information including amino acid and nucleic acid sequences. http://www.ncbi.nlm.nih.gov/gene/205564

Human SENP6 is listed in the NCBI database as Gene ID: 26054. The entry for SENP6 includes information including amino acid and nucleic acid sequences. http://www.ncbi.nlm.nih.gov/gene/26054

Human SENP7 is listed in the NCBI database as Gene ID: 57337. The entry for SENP7 includes information including amino acid and nucleic acid sequences. http://www.ncbi.nlm.nih.gov/gene/57337

An embodiment of the first aspect of the invention is wherein the method additional comprises modulating the activity SETDB1 in the target cells.

SETDB1 is known in the art and information concerning their amino acid sequence and the nucleic acid sequence of the associated genes can be readily identified by the skilled person.

By way of example, human SETDB1 is listed in the NCBI database as Gene ID: 9869. The entry for SETDB1 includes information including amino acid and nucleic acid sequences. http://www.ncbi.nlm.nih.gov/gene/9869

The first aspect of the invention provides a method of preparing a population of iPS cells comprising reducing the amount and/or activity of one or more components of the CAF1 complex, and/or one or more SUMO pathway in a population of target cells, and (ii) optionally isolating the iPS cells from the target cell population, and optionally SETDB1.

A preferred embodiment of the invention is where the step reducing the amount and/or activity of one or more components of the CAF1 complex comprises administering to the cells one or more agents that reduces the expression of CHAF1A, CHAF1B and/or RBBP4.

A preferred embodiment of the invention is where the step reducing the amount and/or activity of one or more components of the SUMO pathway comprises administering to the cells one or more agents that reduces the expression of SUMO1, SUMO2, SUMO3, SUMO4, SAE1, UBA2, PIAS1, PIAS3, PIA3, PIA4, RANBP2, CBX4, NSMCE2, MUL1, HDAC4, HDAC7, TOPORS, FUS, RASd2, TRAF7, SENP1, SENP2, SENP3, SENP5, SENP6 and/or SENP7.

As disclosed in Example 4 and in the accompanying examples, the inventors systematically reduced the expression of example components of the CAF1 complex and SUMO pathway in target cells and measured the effect on iPSC generation. They showed that there was a surprising, and synergistic, effect on the frequency of iPS generation when expression of a components of the CAF1 complex was reduced and also a component of the SUMO pathway.

Hence a preferred embodiment of the first aspect of the invention is wherein the method comprises reducing the amount and/or activity of one or more components of the CAF1 complex and one or more components of the SUMO pathway in a population of target cells. In a further preferred method of the invention the component of the CAF1 complex is a CAF-1 subunit (particularly Chaf1b) and the component of the SUMO pathway is Ube2i.

A preferred embodiment of the invention is where the step reducing the amount and/or activity of one or more components of the SUMO pathway comprises administering to the cells one or more agents that reduces the expression of SETDB1.

A preferred embodiment is where the agent is a siRNA or shRNA molecule.

That method includes the step of reducing the amount and/or activity of the stated target complex or pathway. Individual genetic and protein components of both the complex and pathway are provided above.

There are a number of different means by which the amount or activity of a particular gene or protein can be reduced. These are now discussed below

“Reduction” may be achieved by inhibiting activity of the protein or expression. For purposes of convenience, “reducing activity” will be used herein to refer to reducing activity of components of the CAF1 complex or SUMO pathway (e.g., by causing mRNA degradation, reducing mRNA translation, etc.) or SETDB1.

In some embodiments reducing activity is achieved using RNAi. RNAi is a term well known in the art and is a biological process by which RNA molecules inhibit gene expression, typically by causing the destruction of specific mRNA molecules.

RNAi can be applied to target cells by a number of methods. Typically, a sequence encoding a shRNA (small hairpin RNA molecule) may be expressed intracellularly from an appropriate plasmid, or target cells may be cultured in medium containing siRNA (small interfering RNA). In some embodiments an inhibitor of use in the present invention is an RNAi agent. One of skill in the art will be able to identify an appropriate RNAi agent to inhibit expression of a gene of interest. In some embodiments of the invention, the RNAi agent inhibits expression sufficiently to reduce the average steady state level of the RNA transcribed from the gene (e.g., mRNA) or its encoded protein by, e.g., by at least 50%, 60%, 70%, 80%, 90%, 95%, or more). The RNAi agent may contain a sequence between 15-29 nucleotides long, e.g., 17-23 nucleotides long, e.g., 19-21 nucleotides long, that is 100% complementary to the mRNA or contains up to 1, 2, 3, 4, or 5 nucleotides, or up to about 10-30% nucleotides, that do not participate in Watson-Crick base pairs when aligned with the mRNA to achieve the maximum number of complementary base pairs. The RNAi agent may contain a duplex between 17-29 nucleotides long in which all nucleotides participate in Watson-Crick base pairs or in which up to about 10-30% of the nucleotides do not participate in a Watson-Crick base pair. One of skill in the art will be aware of which sequence characteristics are often associated with superior siRNA functionality and will be aware of algorithms and rules by which such siRNAs can be designed (see, e.g., Jagla, B., et al, RNA, 11(6):864-72, 2005). The methods of the invention can employ siRNAs having such characteristics. In some embodiments the sequence of either or both strands of the RNAi agent is/are chosen to avoid silencing non-target genes, e.g., the strand(s) may have less than 70%, 80%, or 90% complementarity to any mRNA other than the target mRNA. In some embodiments multiple different sequences are used. RNAi agents capable of silencing mammalian genes are commercially available (e.g., from suppliers such as Qiagen, Dharmacon, Ambion/ABI, Sigma-Aldrich, etc.). If multiple iso forms of a gene of interest exist, one can design siRNAs or shRNAs targeted against a region present in all of the isoforms expressed in a given cell of interest.

For the way of guidance to the skilled person, the present application provides at the end of the specification, examples of siRNA sequences which can be used to generate shRNA molecules for the components of the CAF1 complex and SUMO pathway. From this information the skilled person can readily generate appropriate molecules so as to achieve RNAi-mediated reduction in the expression of components of the CAF1 complex and SUMO pathway as used in the method of the invention.

Methods for silencing genes by transfecting cells with siRNA or constructs encoding shRNA are known in the art. To express an RNAi agent in somatic cells, a nucleic acid construct comprising a sequence that encodes the RNAi agent, operably linked to suitable expression control elements, e.g., a promoter, can be introduced into the cells as known in the art. For purposes of the present invention a nucleic acid construct that comprises a sequence that encodes an RNA or polypeptide of interest, the sequence being operably linked to expression control elements such as a promoter that direct transcription in a cell of interest, is referred to as an “expression cassette”. The promoter can be an RNA polymerase I, II, or III promoter functional in somatic mammalian cells. In certain embodiments expression of the RNAi agent is conditional. In some embodiments expression is regulated by placing the sequence that encodes the RNAi agent under control of a regulatable (e.g., inducible or repressible) promoter. The examples provided herein discloses sequences for certain siRNAs that were shown to be effective in inhibiting expression of their target in mouse embryonic fibroblast cells. One of skill in the art will be able to identify siRNA sequences that target corresponding regions of human orthologs.

In a preferred embodiment of the invention, the expression of the agent is transient.

Transient suppression can be achieved through (I) transient delivery methods or (II) stable delivery of inducible/regulatable expression cassettes.

As can be appreciated by the skilled person, examples of transient delivery methods include (1) transient transfection of siRNAs, other inhibitory RNA molecules, (2) transient transfection of DNA or RNA vectors encoding shRNA/siRNA expression cassettes, (3) infection with non-integrating viruses (e.g. AAV, Adenovirus, Sendaivirus and many others) encoding shRNAs/siRNAs or other inhibitory genetic elements to suppress the target.

There are many examples of how to stably deliver inducible/regulatable/conditional expression cassettes in to mammalian cells, e.g retro-/lentiviruses, the CRISPR and TALEN technologies, and other delivery methods. In addition there are many inducible system.

In an embodiment of the invention, the present inventors used a self-inactivating retroviral vector encoding an shRNA under control of a Tet-responsive element promoter (TRE3G). That vector encoded a shRNA to be used in the method of the invention to suppress the expression of one or more components of the CAF1 complex or one or more components of the SUMO pathway in a population of target cells. The vector preferably provides for inducible and reversible expression of the shRNA. As outlined in the accompanying examples, the specific vector is called pSIN-TRE3G-mCherry-miRE-PGK-Neo. However, the skilled person would readily be able to identify suitable transient inducible/regulatable expression cassettes which can be adapted to encode a shRNA molecule to be used in the method of the invention, and also the protocol used to introduce that vector to a population of target cells.

In some embodiments of the invention, cells are contacted with an agent for a time period of at least 1 days while in other embodiments the period of time is at least 3, 5, 10, 15, or 20 days. In some embodiments, cells are contacted for at least 1 and no more than 3, 5, 10, 15, or 20 days.

In certain embodiments of the invention agent is a protein, small molecule, or aptamer. In some embodiments, the agent (e.g., protein, small molecule, or aptamer) binds to and inhibits its target or binds to and inhibits a protein whose activity is needed for the target. Small molecule inhibitors of the target complex or pathway components may be used in various embodiments of the invention.

In some embodiments the concentration of the agent added to the medium is between 10 and 10,000 ng/ml, e.g., between 100 and 5,000 ng/ml, e.g., between 1,000 and 2,500 ng/ml or between 2,500 and 5,000 ng/ml, or between 5,000 and 10,000 ng/ml.

Methods of the invention may include treating the cells with multiple agents either concurrently (i.e., during time periods that overlap at least in part) or sequentially and/or repeating the steps of treating the cells with an agent. The agent used in the repeating treatment may be the same as, or different from, the one used during the first treatment.

The cells may be contacted with a reprogramming agent for varying periods of time. In some embodiments the cells are contacted with the agent for a period of time between 1 hour and 60 days, e.g., between 10 and 30 days, e.g., for about 15-20 days. Reprogramming agents may be added each time the cell culture medium is replaced. The reprogramming agent(s) may be removed prior to performing a selection to enrich for pluripotent cells or assessing the cells for pluripotency characteristics.

In one preferred embodiment of the invention, the agent that reduces the expression of the CAF1 complex component(s) or SUMO pathway component(s) is a RNAi agent which is expressed within a target cell using a transient expression system.

The present inventors have preformed a series of experiments examining different ways in which the expression levels of the identified genes can be modulated in the target cells. As shown in the accompanying Examples, the inventors surprisingly found that it is possible to increase cell reprogramming efficiency using transient expression of RNAi agents which reduce expression of CAF1 complex component(s) or SUMO pathway component(s). In particular, transient suppression of CAF1 (Chaf1a or Chaf1b) and/or Ube2i together with transient expression of OKSM can promote stable reprogramming, even if shRNA/siRNAs and OKSM are expressed for only 2 days. This is a surprising results since established OKSM-based iPSC reprogramming regimens typically require OKSM expression over longer periods of time. As can be appreciated, transient delivery of RNAi agents used in the method of the method of the invention is greatly advantageous over existing methods of modifying target gene expression to promote reprogramming efficiency, since no foreign nucleic acid is permamently incorporated in the target cell genome, and also there is less chance of damaging DNA editing artifacts occurring, as may be the case with CRISPR or other similar technologies.

A further embodiment of the invention is wherein the target cell is exposed to a transient expression system expressing the RNAi agent for 120, 96, 72, 48 or 36 hours. As can be appreciated, the above listed range of hours is not intended to be exhaustive, and merely for expediency all time points between the ranges of the hours provided are included in the scope of the method of the invention.

Therefore a preferred method of the first aspect of the invention is wherein the target cells are administered one or more agents which transiently suppress the expression of the CAF1 complex component(s) and/or SUMO pathway component(s) is a RNAi agents. Methods of transiently suppressing the expression of the CAF1 complex component(s) and/or SUMO pathway component(s) using RNAi agents have been provided above. For example, the self-inactivating retroviral vector encoding an shRNA under control of a Tet-responsive element promoter, described above can be used.

Target cells of use in the invention may be primary cells (non-immortalized cells), such as those freshly isolated from an animal, or may be derived from a cell line capable or prolonged proliferation in culture (e.g., for longer than 3 months) or indefinite proliferation (immortalized cells). Adult somatic cells may be obtained from individuals, e.g., human subjects, and cultured according to standard cell culture protocols available to those of ordinary skill in the art. The cells may be maintained in cell culture following their isolation from a subject. In certain embodiments the cells are passaged once or more following their isolation from the individual (e.g., between 2-5, 5-10, 10-20, 20-50, 50-100 times, or more) prior to their use in a method of the invention. They may be frozen and subsequently thawed prior to use. In some embodiments the cells will have been passaged no more than 1, 2, 5, 10, 20, or 50 times following their isolation from the individual prior to their use in a method of the invention. In some embodiments, methods of the invention utilize cells of a cell line, e.g., a population of largely or substantially identical cells that have typically been derived from a single ancestor cell or from a defined and/or substantially identical population of ancestor cells or from a tissue sample obtained from a particular individual. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

A preferred embodiment of the invention is wherein the target cells are somatic mammalian cells, preferably, human cells, non-human primate cells, or mouse cells.

A preferred embodiment of the invention is wherein the somatic mammalian cells are fibroblasts, adult stem cells, Sertoli cells, granulosa cells, neurons, pancreatic islet cells, epidermal cells, epithelial cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells or skeletal muscle cells.

Somatic cells of use in the present invention are typically mammalian cells, such as, for example, human cells, non-human primate cells, or mouse cells. They may be obtained by well-known methods from various organs, e.g., skin, lung, pancreas, liver, stomach, intestine, heart, reproductive organs, bladder, kidney, urethra and other urinary organs, etc., generally from any organ or tissue containing live somatic cells. Mammalian somatic cells useful in various embodiments of the present invention may be fibroblasts, adult stem cells, Sertoli cells, granulosa cells, neurons, pancreatic islet cells, epidermal cells, epithelial cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells, skeletal muscle cells, etc., generally any nucleated living somatic cells. In some embodiments, the somatic cell is a terminally differentiated cell, i.e., the cell is fully differentiated and does not (under normal conditions in the body) give rise to more specialized cells. In some embodiments the somatic cell is a terminally differentiated cell that does not divide under normal conditions in the body, i.e., the cell cannot self-renew. In some embodiments, the somatic cell is a precursor cell, i.e., the cell is not fully differentiated and is capable of giving rise to cells that are more fully differentiated. In some embodiments, cells that can be obtained relatively convenient procedure from a human subject are used (e.g., fibroblasts, keratinocytes, circulating white blood cells).

In the methods of the present invention the population of target cells may, in general, be cultured under standard conditions of temperature, pH, and other environmental conditions, e.g., as adherent cells in tissue culture plates at 37° C. in an atmosphere containing 5-10% CO₂. The cells and/or the cell culture medium are appropriately modified to achieve reprogramming as described herein. The cell culture medium contains nutrients that are sufficient to maintain viability and, typically, support proliferation of at least some cell types. The medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. Some non-limiting examples are provided herein.

As would be appreciated by the skilled person, the quantity of the agent required to reduce the amount and/or activity of one or more components of the CAF1 complex, and/or one or more components of the SUMO pathway, in a population of target cells, can vary depending on the type of target cell used in the method of the invention. Similarly, the length of time the target cells are exposed to the agents stated above can vary depending on the type of target cell used in the method of the invention.

The quantities and length of time needed to most effectively promote reprogramming in a particular cell type can be readily identified using the methods disclosed herein and also normal experimental procedures. Also, the most effective type of agents can be identified.

For example, the skilled person can perform a series of experiments using the same target cells, then perform the method of the invention using a varying quantity of the said agents for a fixed length of time, and then identify the most effective condition for that target cell type. Similarly, skilled person can perform a series of experiments using the same target cells, then perform the method of the invention using a varying length of time that the cells are exposed to a fixed quantity of the agents, and then identify the most effective condition for that target cell type. Also, similar experiments can performed where the promoter sequences used in the transient expression system are changed, so as to identify the most optimal system. Furthermore, different methods of transfecting the target cells with the transient expression vectors can used so as to also identify the most optimal protocol for the target cells.

As can be appreciated, various routine derivatives of the above approach can be used to best identify the conditions to be applied to a particular target cell type using the claimed method.

In the method of the first aspect of the invention, a population of target cells are cultured in medium suitable for culturing iPS cells while undergoing reprogramming. Exemplary serum-containing iPS medium is made with 80% DMEM (typically KO DMEM), 20% defined fetal bovine serum (FBS) not heat inactivated, 1% non-essential amino acids, 1 mM L-glutamine, and 0.1 mM [beta]-mercaptoethanol. The medium is filtered and stored at 4° C., e.g., for 2 weeks or less. Serum-free ES medium may be prepared with 80% KO DMEM, 20% serum replacement, 1% non-essential amino acids, 1 mM L-glutamine, and 0.1 mM [beta]-mercaptoethanol and a serum replacement such as Invitrogen Cat. No. 10828-028. The medium is filtered and stored at 4° C. Before combining with the cells used for conditioning, human bFGF can be added to a final concentration of 4 ng/mL. StemPro® hESC SFM (Invitrogen Cat. No. A1000701), a fully defined, serum- and feeder-free medium (SFM) specially formulated for the growth and expansion of human embryonic stem cells, is of use. In some embodiments, iPS cells are reprogrammed to one or more differentiated cell types. The iPS cells may be cultured initially in medium suitable for maintaining ES cells and may be transferred to medium suitable for the desired cell type.

The present invention provides a method of preparing a population of iPS cells comprising reducing the amount and/or activity of one or more components of the CAF1 complex, and/or one or more components of the SUMO pathway, in a population of target cells, and (ii) optionally isolating the iPS cells from the target cell population.

The method of the invention is used as part of a reprogramming protocol for the preparation of iPS cells,

“Reprogramming protocol” refers to any treatment or combination of treatments that causes at least some cells to become reprogrammed. In some embodiments, “reprogramming protocol” can refer to a variation of a known reprogramming protocol, wherein a factor or other agent used in a known reprogramming protocol is omitted or modified. In some embodiments, “reprogramming protocol” can refer to a variation of a known reprogramming protocol, wherein a factor or agent known to be of use for reprogramming is used together with a different agent whose utility in reprogramming has not been established.

Details of reprogramming protocols are now provided below.

To reprogram somatic cells to pluripotency, the cells may be treated to cause them to express or contain one or more reprogramming factor or pluripotency factor at levels greater than would be the case in the absence of such treatment. For example, somatic cells may be genetically engineered to express one or more genes encoding one or more such factor(s) and/or may be treated with agent(s) that increase expression of one or more endogenous genes encoding such factors and/or stabilize such factor(s). The agent could be, for example, a small molecule, a nucleic acid, a polypeptide, etc. In some embodiments, pluripotency factors are introduced into somatic cells, e.g., by microinjection or by contacting the cells with the factors under conditions in which the factors are taken up by the cells. In some embodiments the factors are modified to incorporate a protein transduction domain. In some embodiments the cells are permeabilized or otherwise treated to increase their uptake of the factors. Exemplary factors are discussed below.

The transcription factor Oct4 (also called Pou5fl, Oct-3, Oct3/4) is an example of a pluripotency factor. Oct4 has been shown to be required for establishing and maintaining the undifferentiated phenotype of ES cells and plays a major role in determining early events in embryogenesis and cellular differentiation (Nichols et al., 1998, Cell 95:379-391; Niwa et al., 2000, Nature Genet. 24:372-376). Oct4 expression is down-regulated as stem cells differentiate into more specialized cells. Nanog is another example of a pluripotency factor. Nanog is a homeobox-containing transcription factor with an essential function in maintaining the pluripotent cells of the inner cell mass and in the derivation of ES cells from these. Furthermore, overexpression of Nanog is capable of maintaining the pluripotency and self-renewing characteristics of ESCs under what normally would be differentiation-inducing culture conditions. (See Chambers et al., 2003, Cell 113: 643-655; Mitsui et al., Cell. 2003, 1 13(5):631-42). Sox2, another pluripotency factor, is an HMG domain-containing transcription factor known to be essential for normal pluripotent cell development and maintenance (Avilion, A., et al., Genes Dev. 17, 126-140, 2003). Klf4 is a Krüppel-type zinc finger transcription factor initially identified as a Klf family member expressed in the gut (Shields, J. M, et al., J. Biol. Chem. 271:20009-20017, 1996). Overexpression of Klf4 in mouse ES cells was found to prevent differentiation in embryoid bodies formed in suspension culture, suggesting that Klf4 contributes to ES self renewal (Li, Y., et al., Blood 105:635-637, 2005). Sox2 is a member of the family of SOX (sex determining region Y-box) transcription factors and is important for maintaining ES cell self-renewal. c-Myc is a transcription factor that plays a myriad of roles in normal development and physiology as well as being an oncogene whose dysregulated expression or mutation is implicated in various types of cancer (reviewed in Pelengaris S, Khan M., Arch Biochem Biophys. 416(2):129-36, 2003; Cole M D, Nikiforov M A, Curr Top Microbiol Immunol, 302:33-50, 2006). In some embodiments such factors are selected from the group consisting of: Oct4, Sox2, Klf4, and combinations thereof. In some embodiments a different, functionally overlapping Klf family member such as K112 is substituted for Klf4. In some embodiments the factors include at least Oct4. In some embodiments the factors include at least Oct4 and a Klf family member, e.g., Klf2. Lin28 is a developmentally regulated RNA binding protein. In some embodiments somatic cells are treated so that they express or contain one or more reprogramming factors selected from the group consisting of: Oct4, Sox2, Klf4, Nanog, Lin28, and combinations thereof. CCAAT/enhancer-binding-protein-alpha (C/EBPalpha) is another protein that promotes reprogramming at least in certain cell types, e.g., lymphoid cells such as B-lineage cells, is considered a reprogramming factor for such cell types.

In one embodiment, the exogenously introduced gene may be expressed from a chromosomal locus other than the chromosomal locus of an endogenous gene whose function is associated with pluripotency. Such a chromosomal locus may be a locus with open chromatin structure, and contain gene(s) whose expression is not required in somatic cells, e.g., the chromosomal locus contains gene(s) whose disruption will not cause cells to die. Exemplary chromosomal loci include, for example, the mouse ROSA 26 locus and type II collagen (Col2al) locus (See Zambrowicz et al., 1997).

Methods for expressing genes in cells are known in the art. Generally, a sequence encoding a polypeptide or functional RNA such as an RNAi agent is operably linked to appropriate regulatory sequences (e.g., promoters, enhancers and/or other expression control elements). Exemplary regulatory sequences are described in Goeddel; Gene Expression Technology: Methods in Enzymology, Academic Press, San Diego, Calif. (1990). [0086] The gene may be expressed from an inducible or repressible regulatory sequence such that its expression can be regulated. Exemplary inducible promoters include, for example, promoters that respond to heavy metals (CRC Boca Raton, Fla. (1991), 167-220; Brinster et al. Nature (1982), 296, 39-42), to thermal shocks, to hormones (Lee et al. P.N.A.S. USA (1988), 85, 1204-1208; (1981), 294, 228-232; Klock et al. Nature (1987), 329, 734-736; Israel and Kaufman, Nucleic Acids Res. (1989), 17, 2589-2604), promoters that respond to chemical agents, such as glucose, lactose, galactose or antibiotics. A tetracycline-inducible promoter is an example of an inducible promoter that responds to an antibiotic (tetracycline or an analog thereof). See Gossen, M. and Bujard, H., Annu Rev Genet. Vol. 36: 153-173 2002 and references therein. Tetracycline analog includes any compound that displays structural similarity with tetracycline and is capable of activating a tetracycline-inducible promoter. Exemplary tetracycline analogs include, for example, doxycycline, chlorotetracycline and anhydrotetracycline.

In some embodiments of the invention expression of an introduced gene, e.g., a gene encoding a reprogramming factor or RNAi agent is transient. Transient expression can be achieved by transient transfection or by expression from a regulatable promoter. In some embodiments expression can be regulated by, or is dependent on, expression of a site-specific recombinase. Recombinase systems include the Cre-Lox and Flp-Frt systems, among others (Gossen, M. and Bujard, H., 2002). In some embodiments a recombinase is used to turn on expression by removing a stopper sequence that would otherwise separate the coding sequence from expression control sequences. In some embodiments a recombinase is used to excise at least a portion of a gene after reprogramming has been induced. In some embodiments the recombinase is expressed transiently, e.g., it becomes undetectable after about 1-2 days, 2-7 days, 1-2 weeks, etc. In some embodiments the recombinase is introduced from external sources.

It is contemplated that protein reprogramming factors (e.g., Oct4, Sox2, Klf4, etc.) may be introduced into cells, thereby avoiding introducing exogenous genetic material. Such proteins may be modified to include a protein transduction domain. Such uptake-enhancing amino acid sequences are found, e.g., in HIV-I TAT protein, the herpes simplex virus 1 (HSV-I) DNA-binding protein VP22, the Drosophila Antennapedia (Antp) transcription factor, etc. Artificial sequences are also of use. See, e.g., Fischer et al, Bioconjugate Chem., Vol. 12, No. 6, 2001 and U.S. Pat. No. 6,835,810.

It is contemplated that a variety of additional agents may be of use to enhance reprogramming. Such agents may be used in combination with an agent that reduces the amount and/or activity of one or more components of the CAF1 complex, and/or one or more components of the SUMO pathway, for example the shRNA agents disclosed herein.

While the present disclosure has focused on reprogramming somatic cells to pluripotency, the inventive methods may be applied to reprogram differentiated somatic cells from a first cell type to a second cell type. For example, it is contemplated that modulating genes and processes identified herein will enhance reprogramming protocols that involve expressing particular combinations of transcription factors in cells to convert them into cells of a different type. Such reprogramming protocols involving modulation of targets identified herein.

In certain embodiments the cells are cultured on or in the presence of a material that mimics one or more features of the extracellular matrix or comprises one or more extracellular matrix or basement membrane components. In some embodiments Matrigel™ is used. Other materials include proteins or mixtures thereof such as gelatin, collagen, fibronectin, etc. In certain embodiments of the invention the cells are cultured in the presence of a feeder layer of cells. Such cells may, for example, be of murine or human origin. They may be irradiated, chemically inactivated by treatment with a chemical inactivator such as mitomycin c, or otherwise treated to inhibit their proliferation if desired. In other embodiments the target cells are cultured without feeder cells.

The iPS cells prepared according to the method of the first aspect of the invention may be assessed for one or more characteristics of a desired cell state or cell type. For example, cells may be assessed for pluripotency characteristic(s). The presence of pluripotency characteristic(s) indicates that the target cells have been reprogrammed to a pluripotent state. The term “pluripotency characteristics”, as used herein, refers to characteristics associated with and indicative of pluripotency, including, for example, the ability to differentiate into cells derived from all three embryonic germ layers all types and a gene expression pattern distinct for a pluripotent cell, including expression of pluripotency factors and expression of other ES cell markers.

To assess potentially reprogrammed target cells for pluripotency characteristics, one may analyze such cells for particular growth characteristics and ES cell-like morphology. Cells may be injected subcutaneously into immunocompromised SCID mice to determine whether they induce teratomas (a standard assay for ES cells). ES-like cells can be differentiated into embryoid bodies (another ES specific feature). Moreover, ES-like cells can be differentiated in vitro by adding certain growth factors known to drive differentiation into specific cell types. Self-renewing capacity, marked by induction of telomerase activity, is another plutipotency characteristic that can be monitored. One may carry out functional assays of the reprogrammed target cells by introducing them into blastocysts and determining whether the cells are capable of giving rise to all cell types. See Hogan et al., 2003. If the reprogrammed cells are capable of forming a few cell types of the body, they are multipotent; if the reprogrammed cells are capable of forming all cell types of the body including germ cells, they are pluripotent.

One may also examine the expression of an individual pluripotency factor. Additionally or alternately, one may assess expression of other ES cell markers such as stage-specific embryonic 1 5 antigens-1, -3, and -4 (SSEA-1, SSEA-3, SSEA-4), which are glycoproteins specifically expressed in early embryonic development and are markers for ES cells (Solter and Knowles, 1978, Proc. Natl. Acad. Sci. USA 75:5565-5569; Kannagi et al., 1983, EMBO J 2:2355-2361). Elevated expression of the enzyme alkaline phosphatase (AP) is another marker associated with undifferentiated embryonic stem cells (Wobus et al., 1984, Exp. Cell 152:212-219; Pease et al., 1990, Dev. Biol. 141:322-352). Additional ES cell markers are described in Ginis, L, et al., Dev. Biol, 269: 369-380, 2004 and in The International Stem Cell Initiative, Adewumi 0, et al., Nat Biotechnol., 25(7):803-16, 2007 and references therein. For example, TRA-1-60, TRA-1-81, GCTM2 and GCT343, and the protein antigens CD9, Thy1 (CD90), class 1 HLA, NANOG, TDGF1, DNMT3B, GABRB3 and GDF3, REX-I, TERT, UTF-I, TRF-I, TRF-2, connexin43, connexin45, Foxd3, FGFR-4, ABCG-2, and Glut-1 are of use.

One may perform expression profiling of the reprogrammed target cells to assess their pluripotency characteristics. Pluripotent cells, such as embryonic stem cells, and multipotent cells, such as adult stem cells, are known to have a distinct pattern of global gene expression. See, for example, Ramalho-Santos et al., Science 298: 597-600, 2002; Ivanova et al., Science 298: 601-604, 2002; Boyer, L A, et al. Nature 441, 349, 2006, and Bernstein, B E, et al., Cell 125 (2), 315, 2006. One may assess DNA methylation, gene expression, and/or epigenetic state of cellular DNA, and/or developmental potential of the cells, e.g., as described in Wernig, M., et al., Nature, 448:318-24, 2007. Cells that are able to form teratomas containing cells having characteristics of endoderm, mesoderm, and ectoderm when injected into SCID mice and/or possess ability to participate (following injection into murine blastocysts) in formation of chimeras that survive to term are considered pluripotent. Another method of use to assess pluripotency is determining whether the cells have reactivated a silent X chromosome.

Similar methods may be used to assess efficiency of reprogramming cells to a desired cell type or lineage. Expression of markers that are selectively or specifically expressed in such cells may be assessed. For example, markers expressed selectively or specifically by neural, hematopoietic, myogenic, or other cell lineages and differentiated cell types are known, and their expression can be assessed. In some embodiments of the invention the expression level of 2-5, 5-10, 10-25, 25-50, 50-100, 100-250, 250-500, 500-1000, or more RNAs (e.g., mRNAs) or proteins is increased by reprogramming the cell according to the methods of the invention. Functional or morphological characteristics of the cells can be assessed to evaluate the efficiency of reprogramming.

Certain methods of the invention include a step of identifying or selecting cells that express a marker that is expressed by multipotent or pluripotent cells or by cells of a desired cell type or lineage. Standard cell separation methods, e.g., flow cytometry, affinity separation, etc. may be used. Alternately or additionally, one could select cells that do not express markers characteristic of the cells from which the potentially reprogrammed cells were derived. Other methods of separating cells may utilize differences in average cell size or density that may exist between pluripotent cells and original target cells. For example, cells can be filtered through materials having pores that will allow only certain cells to pass through.

In some embodiments the target cells contain a nucleic acid comprising regulatory sequences of a gene encoding a pluripotency factor operably linked to a selectable or detectable marker (e.g., GFP or neo). The nucleic acid sequence encoding the marker may be integrated at the endogenous locus of the gene encoding the pluripotency factor (e.g., Oct4, Nanog) or the construct may comprise regulatory sequences operably linked to the marker. Expression of the marker may be used to select, identify, and/or quantify reprogrammed cells.

Any of the methods of the invention that relate to generating a reprogrammed target cell may include a step of obtaining a target cell or obtaining a population of target cells from an individual in need of cell therapy. iPS are generated, selected, or identified from among the obtained cells or cells descended from the obtained cells. Optionally the cell(s) are expanded in culture prior to generating, selecting, or identifying iPS cells genetically matched to the donor.

In some embodiments colonies are subcloned and/or passaged once or more in order to obtain a population of cells enriched for desired cells, i.e iPS cells. The enriched population may contain at least 95%, 96%, 97%, 98%, 99% or more, e.g., 100% cells of a desired type. The invention provides cell lines of target cells that have been stably and heritably reprogrammed to an ES-like state.

In some embodiments, the methods employ morphological criteria to identify reprogrammed cells from among a population of cells that are not reprogrammed to a desired type. In some embodiments, the methods employ morphological criteria to identify target cells that have been reprogrammed to an ES-like state from among a population of cells that are not reprogrammed or are only partly reprogrammed to an ES-like state. “Morphological criteria” is used in a broad sense to refer to any visually detectable feature or characteristic of the cells or colonies. Morphological criteria include, e.g., the shape of the colonies, the sharpness of colony boundaries, the density, small size, and rounded shape of the cells relative to non-reprogrammed cells, etc. For example, dense colonies composed of small, rounded cells, and having sharp colony boundaries are characteristic of ES and iPS cells. The invention encompasses identifying and, optionally, isolating colonies (or cells from colonies) wherein the colonies display one or more characteristics of a desired cell type. The iPS cells may be identified as colonies growing in a first cell culture dish (which term refers to any vessel, plate, dish, receptacle, container, etc, in which living cells can be maintained in vitro) and the colonies, or portions thereof, transferred to a second cell culture dish, thereby isolating reprogrammed cells. The cells may then be further expanded.

The present invention provides iPS cells produced by the methods of the invention. These cells have numerous applications in medicine, agriculture, and other areas of interest. The invention provides methods for the treatment or prevention of a condition in a mammal. In one embodiment, the methods involve obtaining somatic cells from the individual, using these to prepare a target cell population, and preparing a population of iPS cells according to the claimed invention.

In certain embodiments of the invention the obtained iPS cells are then cultured under conditions suitable for their development into cells of a desired cell type, i.e. they then become re-differentiated iPS cells. The cells of the desired cell type are introduced into the individual to treat the condition. The iPS cells can also be induced to develop a desired organ, which is harvested and introduced into the individual to treat the condition. The condition may be any condition in which cell or organ function is abnormal and/or reduced below normal levels. Thus the invention encompasses obtaining somatic cells from an individual in need of cell therapy, using these cells as the target cell population in the claimed method, and optionally differentiating iPS cells to generate cells of one or more desired cell types, and introducing the cells into the individual. An individual in need of cell therapy may suffer from any condition, wherein the condition or one or more symptoms of the condition can be alleviated by administering cells to the donor and/or in which the progression of the condition can be slowed by administering cells to the individual. The method may include a step of identifying or selecting reprogrammed somatic cells and separating them from cells that are not reprogrammed.

The iPS cells, and thus may be induced to differentiate to obtain the desired cell types according to known methods to differentiate such cells. For example, the iPS cells may be induced to differentiate into hematopoietic stem cells, muscle cells, cardiac muscle cells, liver cells, pancreatic cells, cartilage cells, epithelial cells, urinary tract cells, nervous system cells (e.g., neurons) etc., by culturing such cells in differentiation medium and under conditions which provide for cell differentiation. Medium and methods which result in the differentiation of embryonic stem cells obtained using traditional methods are known in the art, as are suitable culturing conditions. Such methods and culture conditions may be applied to the iPS cells obtained according to the present invention. See, e.g., Trounson, A., The production and directed differentiation of human embryonic stem cells, Endocr Rev. 27(2):208-19, 2006 and references therein, all of which are incorporated by reference, for some examples. See also Yao, S., et al, Long-term self-renewal and directed differentiation of human embryonic stem cells in chemically defined conditions, Proc Natl Acad Sci USA, 103(18): 6907-6912, 2006 and references therein, all of which are incorporated by reference.

Thus, using known methods and culture medium, one skilled in the art may culture iPS cells to obtain desired differentiated cell types, e.g., neural cells, muscle cells, hematopoietic cells, etc. The subject cells may be used to obtain any desired differentiated cell type. Such differentiated human cells afford a multitude of therapeutic opportunities. For example, human hematopoietic stem cells derived from cells reprogrammed according to the present invention may be used in medical treatments requiring bone marrow transplantation. Such procedures are used to treat many diseases, e.g., late stage cancers and malignancies such as leukemia. Such cells are also of use to treat anemia, diseases that compromise the immune system such as AIDS, etc. The methods of the present invention can also be used to treat, prevent, or stabilize a neurological disease such as Alzheimer's disease, Parkinson's disease, Huntington's disease, or ALS, lysosomal storage diseases, multiple sclerosis, or a spinal cord injury. For example, somatic cells may be obtained from the individual in need of treatment, and reprogrammed to gain pluripotency, and cultured to derive neurectoderm cells that may be used to replace or assist the normal function of diseased or damaged tissue.

Re-diffentiated iPS cells that produce a growth factor or hormone such as insulin, etc., may be administered to a mammal for the treatment or prevention of endocrine disorders. Re-diffentiated iPS cells that form epithelial cells may be administered to repair damage to the lining of a body cavity or organ, such as a lung, gut, exocrine gland, or urogenital tract. It is also contemplated that iPS may be administered to a mammal to treat damage or deficiency of cells in an organ such as the bladder, brain, esophagus, fallopian tube, heart, intestines, gallbladder, kidney, liver, lung, ovaries, pancreas, prostate, spinal cord, spleen, stomach, testes, thymus, thyroid, trachea, ureter, urethra, or uterus.

iPS cells may be combined with a matrix to form a tissue or organ in vitro or in vivo that may be used to repair or replace a tissue or organ in a recipient mammal (such methods being encompassed by the term “cell therapy”). For example, iPS cells may be cultured in vitro in the presence of a matrix to produce a tissue or organ of the urogenital, cardiovascular, or musculoskeletal system. Alternatively, a mixture of the cells and a matrix may be administered to a mammal for the formation of the desired tissue in vivo. The iPS cells produced according to the invention may be used to produce genetically engineered or transgenic differentiated cells, e.g., by introducing a desired gene or genes, or removing all or part of an endogenous gene or genes of iPS cells produced according to the invention, and allowing such cells to differentiate into the desired cell type. One method for achieving such modification is by homologous recombination, which technique can be used to insert, delete or modify a gene or genes at a specific site or sites in the genome.

This methodology can be used to replace defective genes or to introduce genes which result in the expression of therapeutically beneficial proteins such as growth factors, hormones, lymphokines, cytokines, enzymes, etc. For example, the gene encoding brain derived growth factor maybe introduced into human embryonic or stem-like cells, the cells differentiated into neural cells and the cells transplanted into a Parkinson's patient to retard the loss of neural cells during such disease. Using known methods to introduced desired genes/mutations into iPS cells, the iPS cells may be genetically engineered, and the resulting engineered cells differentiated into desired cell types, e.g., hematopoietic cells, neural cells, pancreatic cells, cartilage cells, etc. Genes which may be introduced into the iPS cells include, for example, epidermal growth factor, basic fibroblast growth factor, glial derived neurotrophic growth factor, insulin-like growth factor (I and II), neurotrophin3, neurotrophin-4/5, ciliary neurotrophic factor, AFT-1, cytokine genes (interleukins, interferons, colony stimulating factors, tumor necrosis factors (alpha and beta), etc.), genes encoding therapeutic enzymes, collagen, human serum albumin, etc.

Negative selection systems known in the art can be used for eliminating therapeutic cells from a patient if desired. For example, cells transfected with the thymidine kinase (TK) gene will lead to the production of reprogrammed cells containing the TK gene that also express the TK gene. Such cells may be selectively eliminated at any time from a patient upon gancyclovir administration. Such a negative selection system is described in U.S. Pat. No. 5,698,446. In other embodiments the cells are engineered to contain a gene that encodes a toxic product whose expression is under control of an inducible promoter. Administration of the inducer causes production of the toxic product, leading to death of the cells. Thus any of the somatic cells of the invention may comprise a suicide gene, optionally contained in an expression cassette, which may be integrated into the genome. The suicide gene is one whose expression would be lethal to cells. Examples include genes encoding diphtheria toxin, cholera toxin, ricin, etc. The suicide gene may be under control of expression control elements that do not direct expression under normal circumstances in the absence of a specific inducing agent or stimulus. However, expression can be induced under appropriate conditions, e.g., (i) by administering an appropriate inducing agent to a cell or organism or (ii) if a particular gene (e.g., an oncogene, a gene involved in the cell division cycle, or a gene indicative of dedifferentiation or loss of differentiation) is expressed in the cells, or (iii) if expression of a gene such as a cell cycle control gene or a gene indicative of differentiation is lost. See, e.g., U.S. Pat. No. 6,761,884. In some embodiments the gene is only expressed following a recombination event mediated by a site-specific recombinase. Such an event may bring the coding sequence into operable association with expression control elements such as a promoter. Expression of the suicide gene may be induced if it is desired to eliminate cells (or their progeny) from the body of a subject after the cells (or their ancestors) have been administered to a subject. For example, if a reprogrammed somatic cell gives rise to a tumor, the tumor can be eliminated by inducing expression of the suicide gene. In some embodiments tumor formation is inhibited because the cells are automatically eliminated upon dedifferentiation or loss of proper cell cycle control.

Examples of diseases, disorders, or conditions that may be treated or prevented include neurological, endocrine, structural, skeletal, vascular, urinary, digestive, integumentary, blood, immune, auto-immune, inflammatory, endocrine, kidney, bladder, cardiovascular, cancer, circulatory, digestive, hematopoietic, and muscular diseases, disorders, and conditions. In addition, reprogrammed cells may be used for reconstructive applications, such as for repairing or replacing tissues or organs. In some embodiments, it may be advantageous to include growth factors and proteins or other agents that promote angiogenesis. Alternatively, the formation of tissues can be effected totally in vitro, with appropriate culture media and conditions, growth factors, and biodegradable polymer matrices.

The present invention contemplates all modes of administration, including intramuscular, intravenous, intraarticular, intralesional, subcutaneous, or any other route sufficient to provide a dose adequate to prevent or treat a disease. The iPS cells may be administered to the mammal in a single dose or multiple doses. When multiple doses are administered, the doses may be separated from one another by, for example, one week, one month, one year, or ten years. One or more growth factors, hormones, interleukins, cytokines, or other cells may also be administered before, during, or after administration of the cells to further bias them towards a particular cell type.

The iPS cells obtained using methods of the present invention may be used as an in vitro model of differentiation, e.g., for the study of genes which are involved in the regulation of early development. Differentiated cell tissues and organs generated using the reprogrammed cells may be used to study effects of drugs and/or identify potentially useful pharmaceutical agents.

The reprogramming methods disclosed herein may be used to generate iPS cells, for a variety of animal species. The iPS cells generated can be useful to produce desired animals. Animals include, for example, avians and mammals as well as any animal that is an endangered species. Exemplary birds include domesticated birds (e.g., chickens, ducks, geese, turkeys). Exemplary mammals include murine, caprine, ovine, bovine, porcine, canine, feline and non-human primate. Of these, preferred members include domesticated animals, including, for examples, cattle, pigs, horses, cows, rabbits, guinea pigs, sheep, and goats.

Hence a further aspect of the invention provides a method for preparing a population of differentiated cells, comprising (i) preparing a population of iPS cells according to the method of the first aspect of the invention, (ii) differentiating the iPS cells using a protocol or factor to form a population of differentiated cells. Methods for reprogramming the cells and their utility are provided above.

A further aspect of the invention provides a population of iPS cells prepared according to the method of the first aspect of the invention.

A further aspect of the invention provides cell culture media comprising one or more agents that inhibit the expression of CHAF1A, CHAF1B and/or RBBP4, and/or one or more agents that inhibit the expression of SUMO1, SUMO2, SUMO3, SUMO4, SAE1, UBA2, PIAS1, PIAS3, PIA3, PIA4, RANBP2, CBX4, NSMCE2, MUL1, HDAC4, HDAC7, TOPORS, FUS, RASd2, TRAF7, SENP1, SENP2, SENP3, SENP5, SENP6 and/or SENP7.

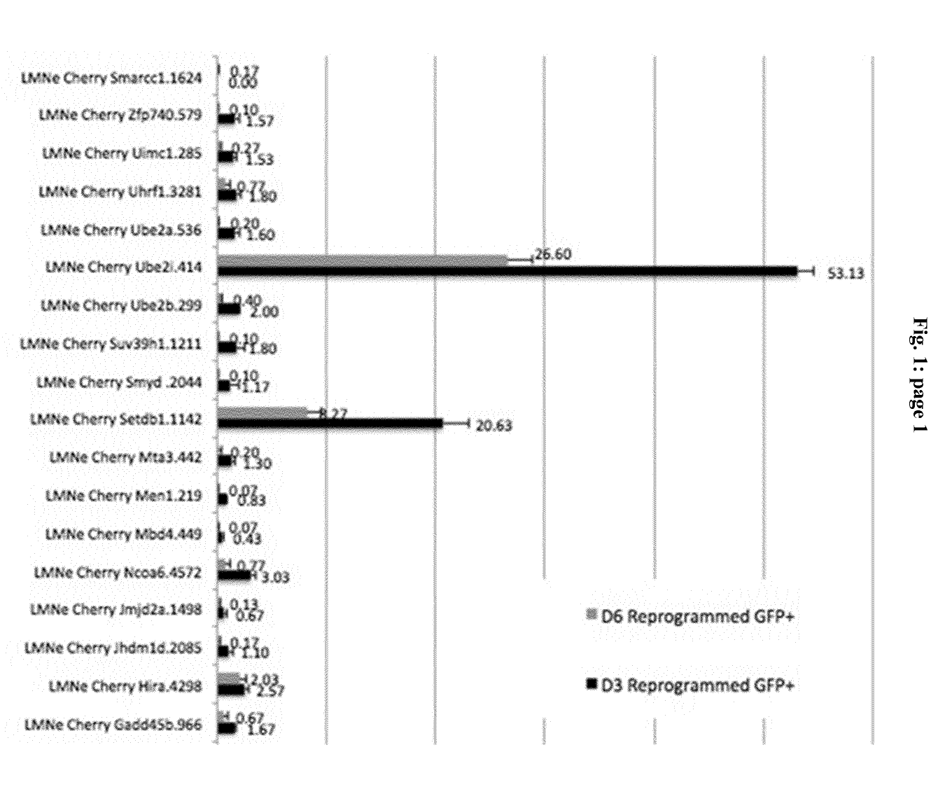

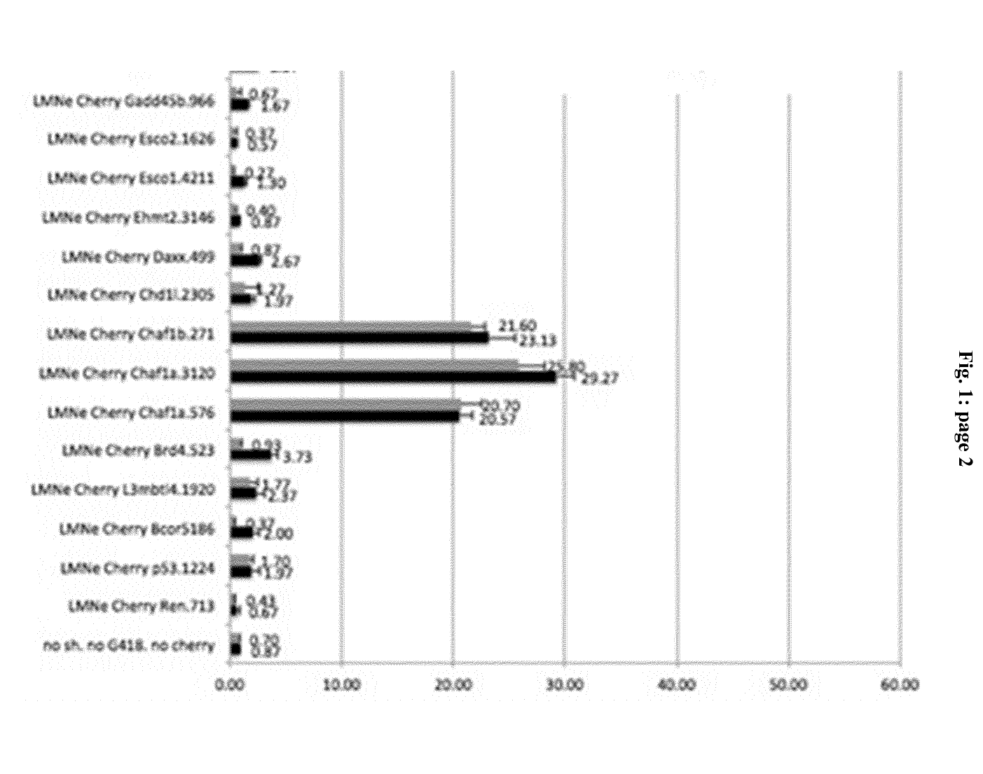

FIG. 1: Percentage of Oct4-GFP positive cells at day 11 after doxycycline induction (=D0). Hairpins were transduced day −3 (D3) and −6 (D6) relative to OSKM induction. Medium: DMEM with FCS and ascorbate

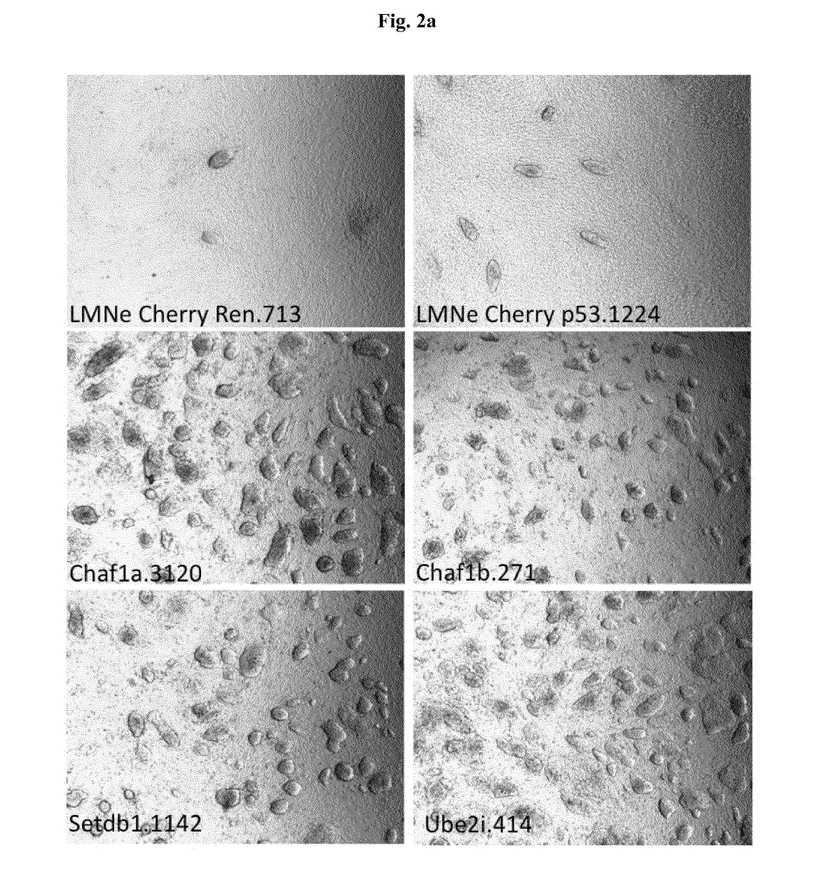

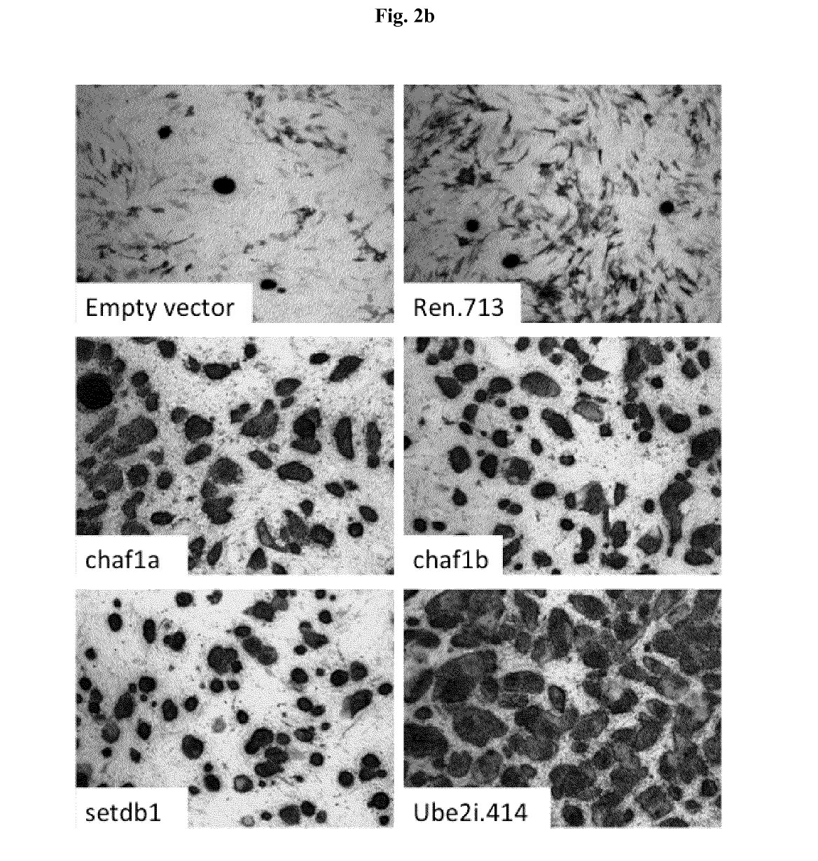

FIG. 2: (a) RNAi knockdown of top scoring reprogramming roadblocks. Images show effects of one shRNA for each top scoring gene on iPS colony formation. Controls include a neutral shRNA (Ren.713) and an shRNA targeting Trp53 (a known reprogramming roadblock). iPS cells appear as dome shaped colonies on a layer of not or partially reprogrammed MEFs. (b) Appearance of iPS cells split 1:5 at day 11 and grown for an additional 3 days in FCS and LIF containing medium. Very dark/black clusters are indicative of iPS formation in alkaline phosphatase staining.

FIG. 3: Percentage of Oct4-GFP positive cells during OSKM induced reprogramming. Hairpins were transduced day −3 (D3) relative to OSKM induction. Medium: DMEM with FCS and ascorbate. Note that knockdown of Caf-1 complex members leads to a dramatically accelerated expression of the Oct4-GFP reporter. Ube2i knockdown leads to minimal advance in iPS formation, however a rapid increase at later timepoints. While GFP+ cell percentage in Caf-1 knockdown saturates and only increases in a second growth phase.

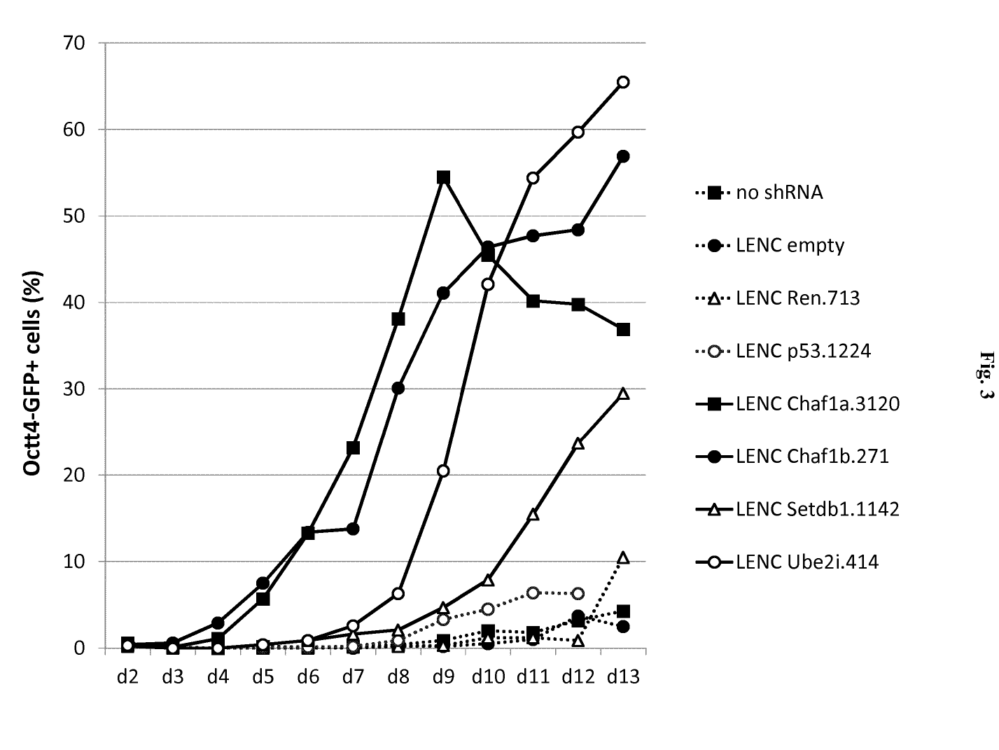

FIG. 4: Percentage of Oct4-GFP positive cells at day 13 after doxycycline induction (=D0). Hairpins were transduced day −3 (D3) and −6 (D6) relative to OSKM induction. Medium: DMEM with FCS and ascorbate. Cells were split and doxycycline was removed from culture condition at indicated days. All conditions were set off doxycycline the latest at day 7.

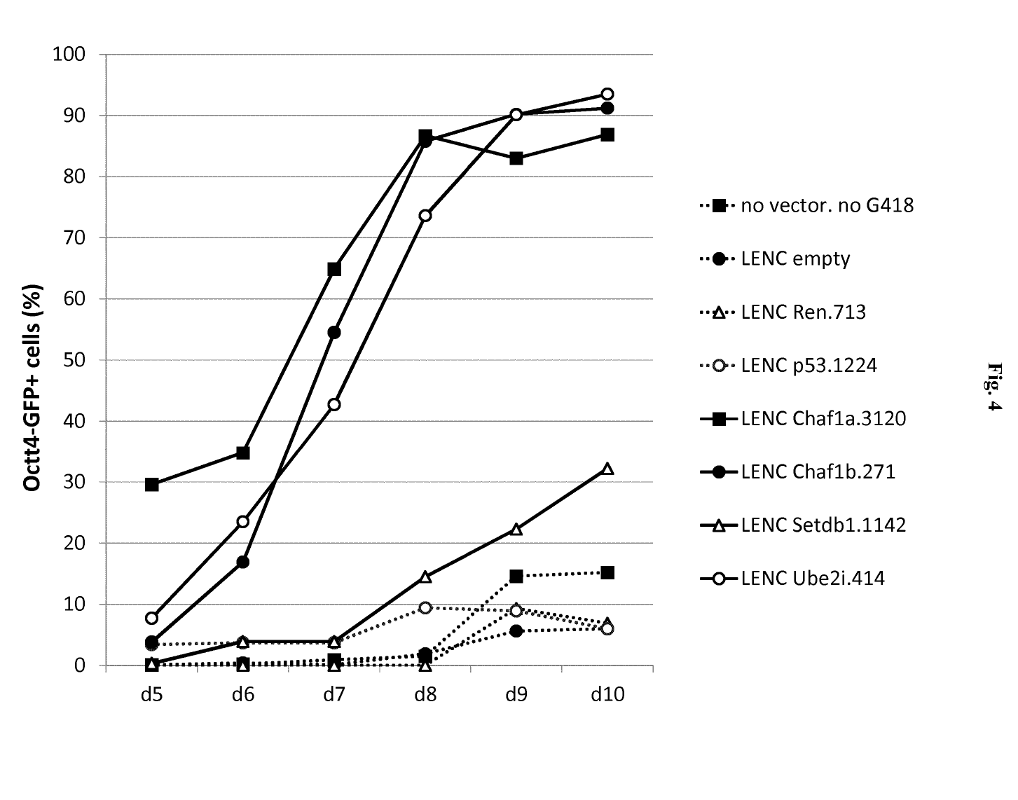

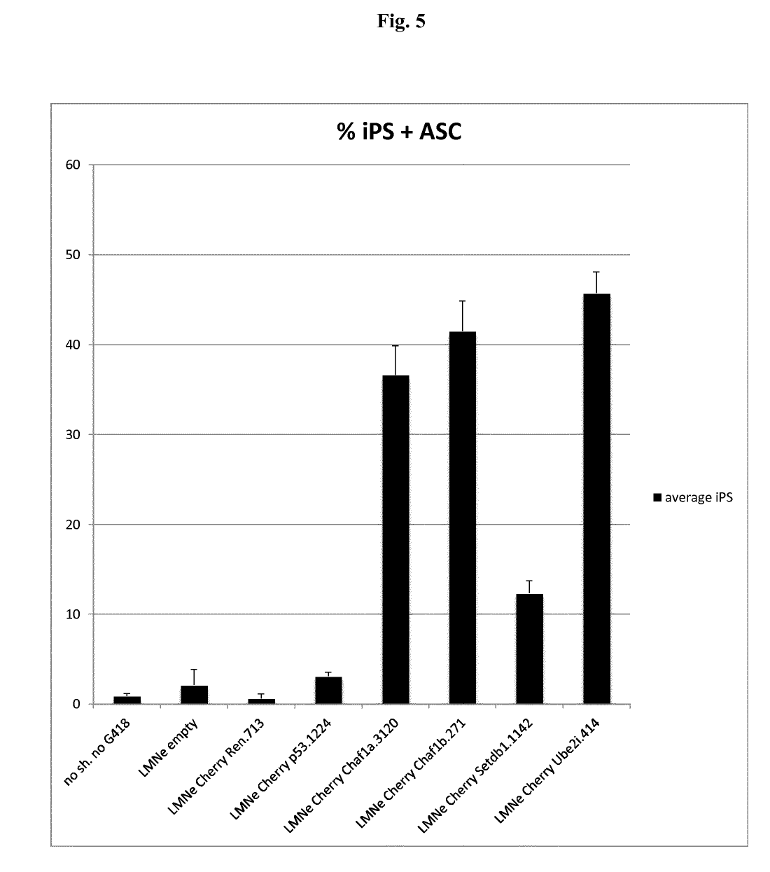

FIG. 5: Percentage of Oct4-GFP positive cells at day 11 after doxycycline induction (=D0). Hairpins were transduced day −3. Medium: DMEM with FCS and ascorbate.

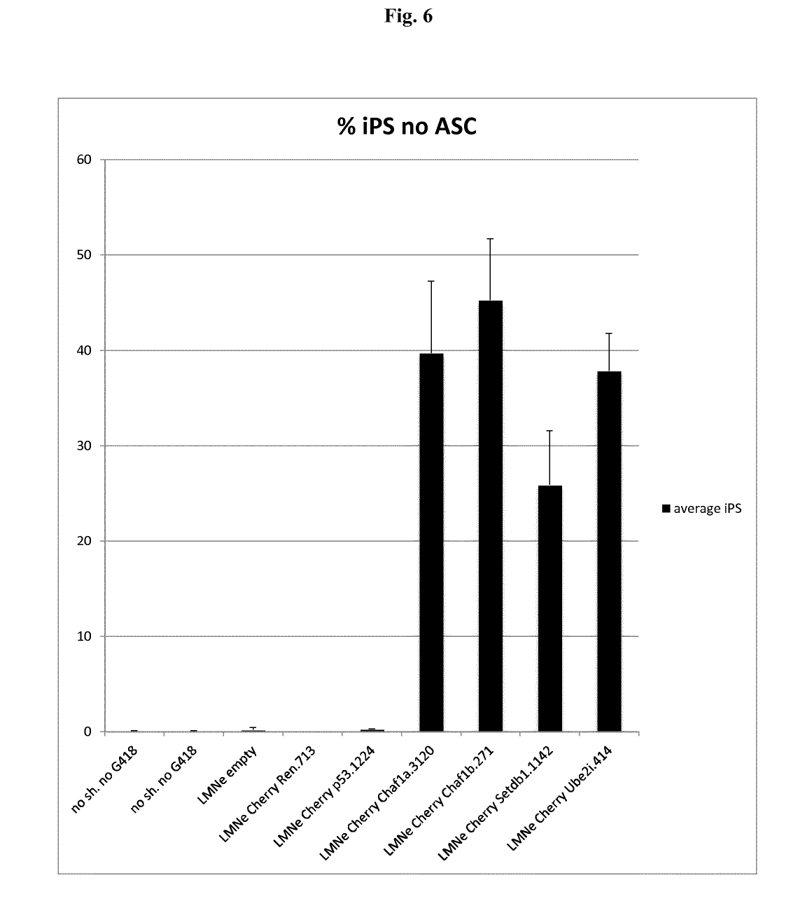

FIG. 6: Percentage of Oct4-GFP positive cells at day 11 after doxycycline induction (=D0). Hairpins were transduced day −3. Medium: DMEM with FCS, without ascorbate

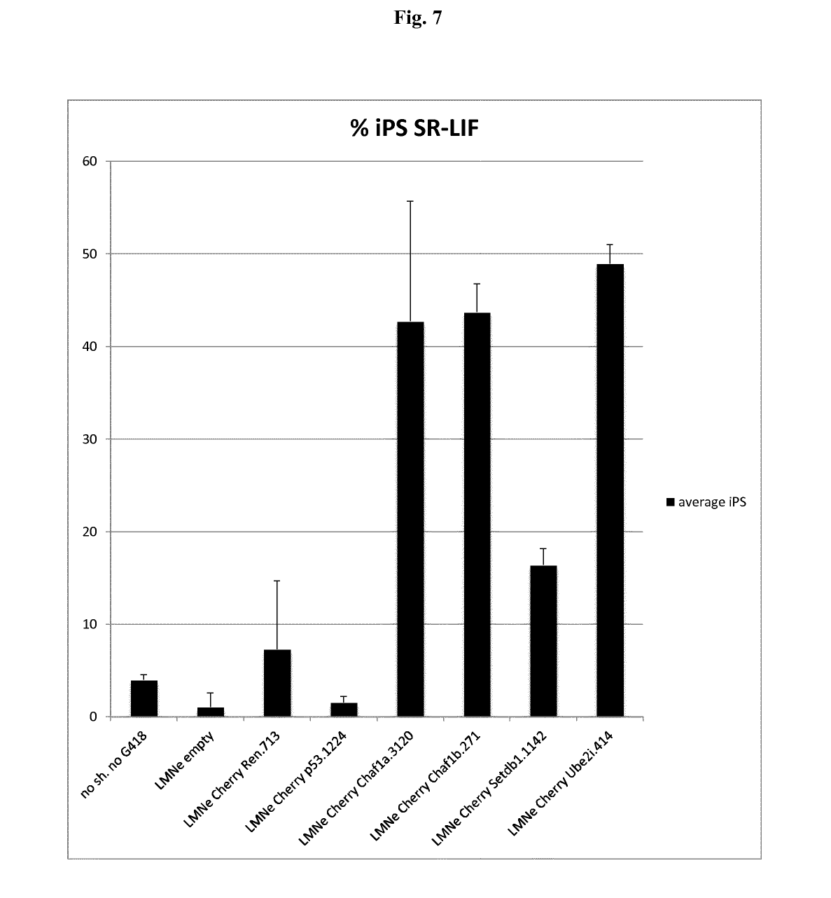

FIG. 7: Percentage of Oct4-GFP positive cells at day 11 after doxycycline induction (=D0). Hairpins were transduced day −3. Medium: DMEM with serum replacement and minimal FCS, without ascorbate

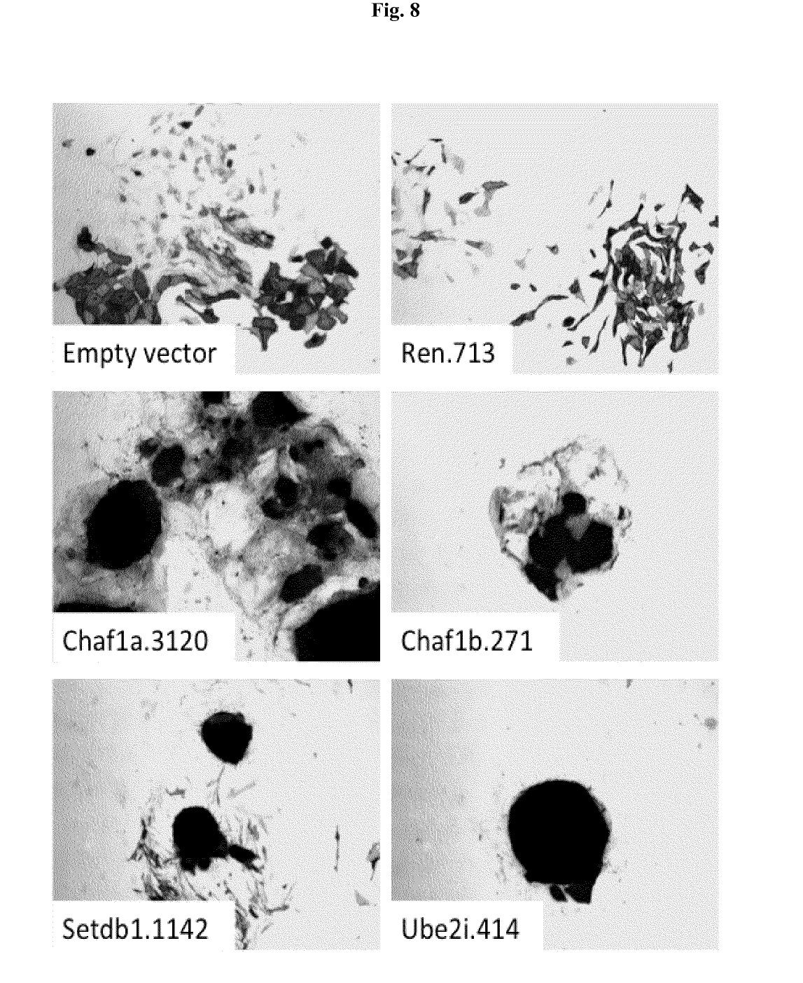

FIG. 8: Appearance of single cell derived iPS cell colonies grown in serum replacement in presence of 2i. Very dark/black clusters are indicative of iPS formation in alkaline phosphatase staining. Note that colonies in Chaf1a/b knockdown appear from a cluster of pre-iPS cells, while colonies in Setdb1 and in particular Ube2i appear to form in absence of pre-iPS clusters.

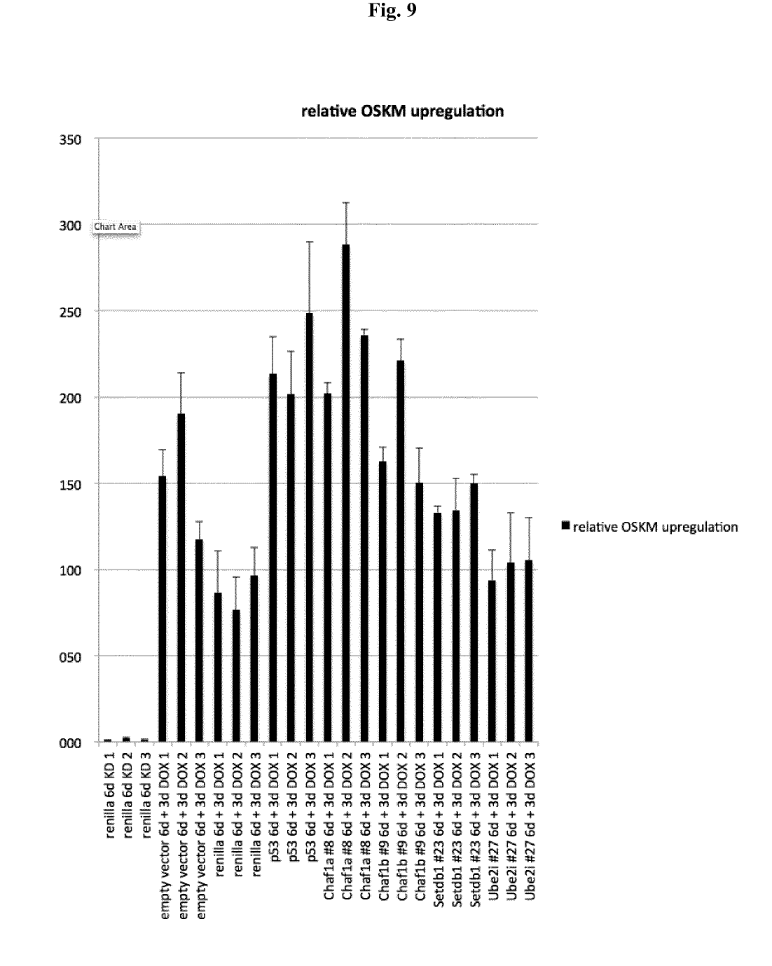

FIG. 9 Expression level of OSKM cassette quantified by Q-PCR and normalized to actin. Primers used were specific to OSKM cassette: Oct4-3′out: CCCACTTCACCACACTCTACTCAGTC (SEQ ID NO:1; Klf4-5′out: GCTGGACGCAGTGTCTTCTCCCTTCC (SEQ ID NO:2). No expression of OSKM is arbitrarily set to 1. PCR was done in triplicate each on 3 biological triplicates to account for intrinsic variation. No major differences in induced expression level can be detected.

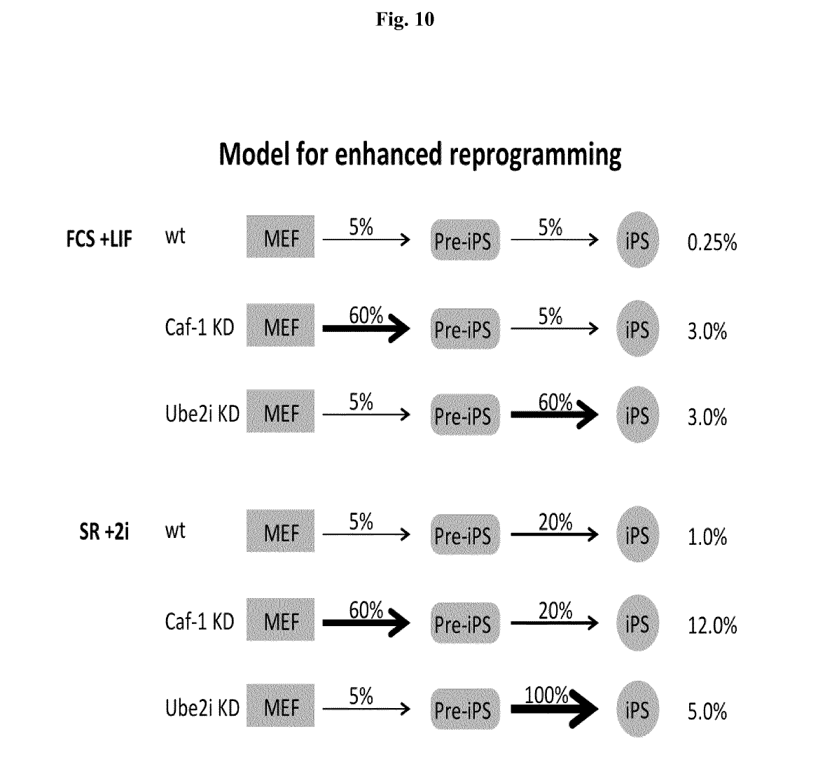

FIG. 10. Model for enhanced reprogramming.

Inhibition of CAF1 may improve the transition of differentiated cells towards pre-iPS cells while inhibition of SUMOylation may enhance the transition of pre-iPS cells towards iPS cells.

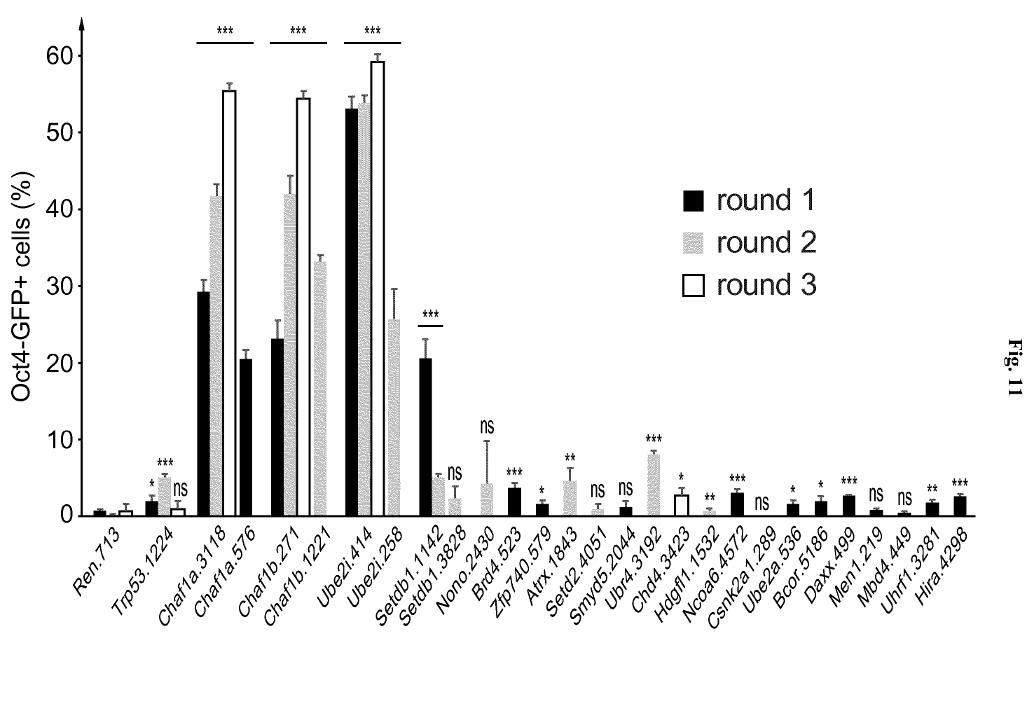

FIG. 11: Validation of hits from the multiplex screening strategy. Error bars indicate standard deviation (SD) from biological triplicates. Star (*) denotes statistically significant differences.

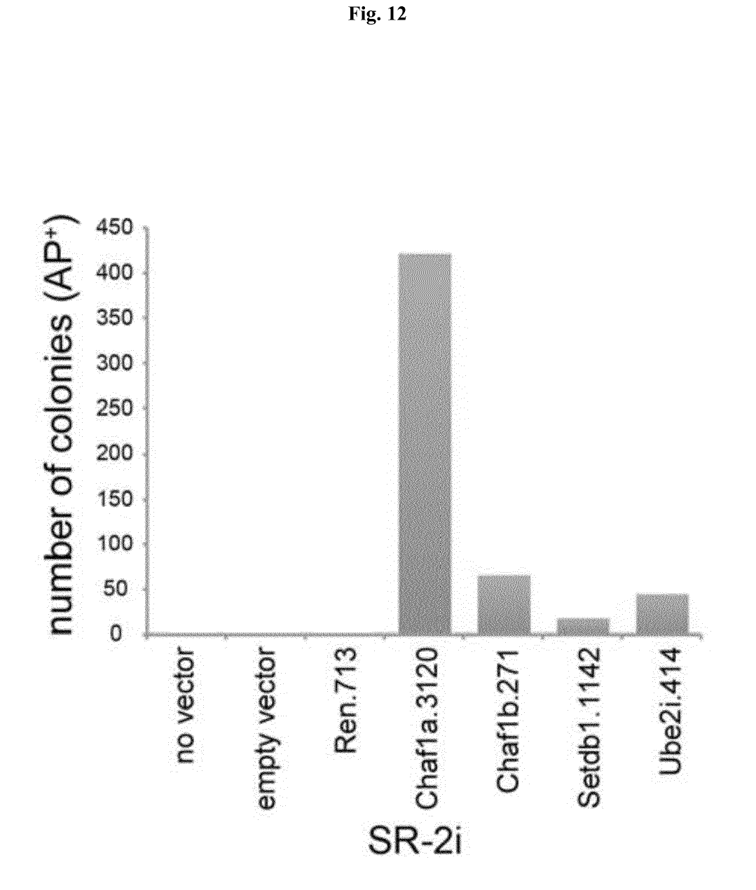

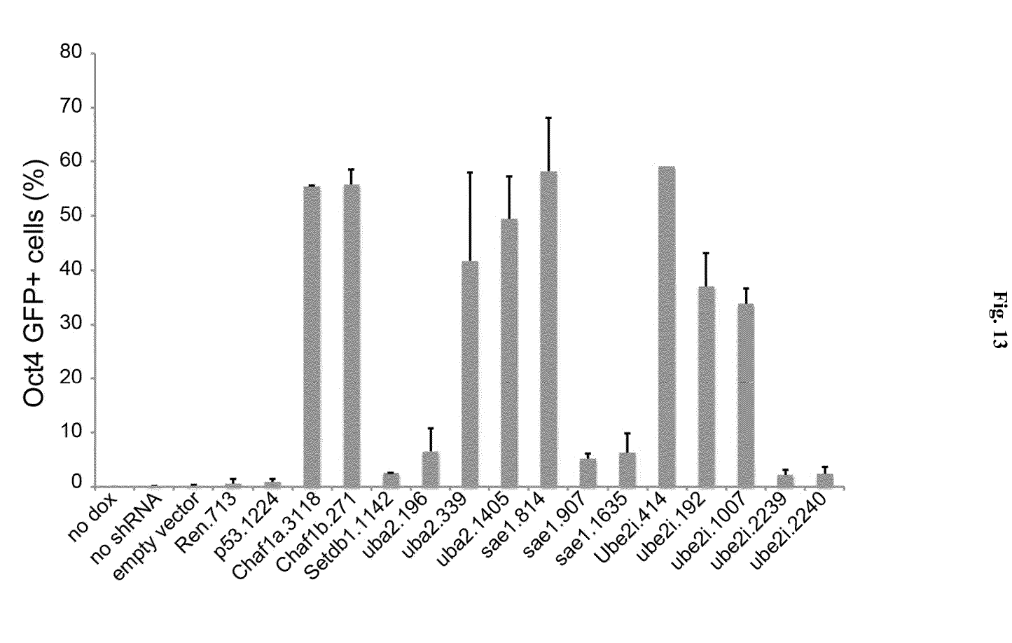

FIG. 12: Number of dox-independent colonies emerging from 10,000 MEFs carrying shRNA vectors against indicated targets in serum replacement media containing 2i.

FIG. 13: Effect of suppressing SUMO E2 ligase Ube2i, E1 ligases Sae1 and Uba2 on formation on reprogramming. Shown is fraction of Oct4-GFP+ cells at day 11 (7 days of OKSM induction, 4 days of transgene-independent growth). Error bars are STDEV on biological triplicate. Suppression of various enzymes in SUMOylation pathways strongly enhances iPS reprogramming.

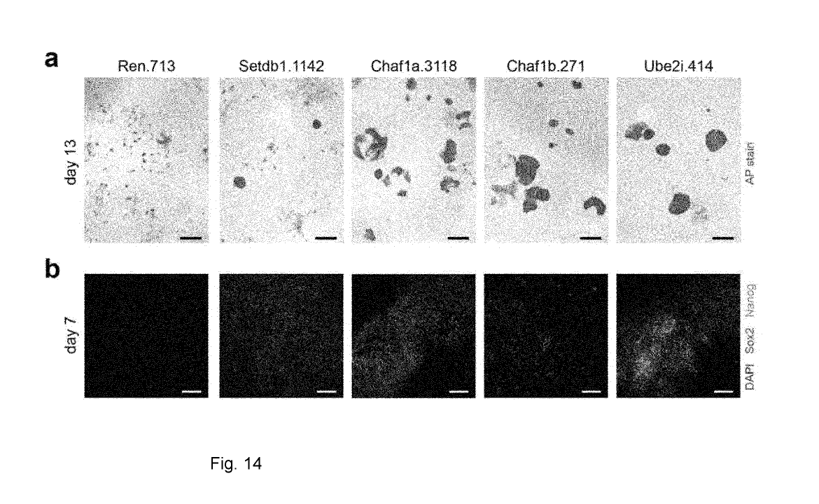

FIG. 14: Phenotypic analysis of iPS cells generated through enhanced reprogramming after suppression of Chaf1a, Chaf1b or Ube2i (a) Alkaline phosphatase staining of iPSCs emerging at day 13 upon knockdown using the indicated shRNAs (scale bar 1 mm). (b) Immunofluorescence staining for Nanog and Sox2 expression in colonies emerging seven days after expression of OKSM in the presenence of the indicated shRNAs. Colonies forming after suppression of Chaf1a, Chaf1b Ube2i express Nanog (scale bar 100 μm).

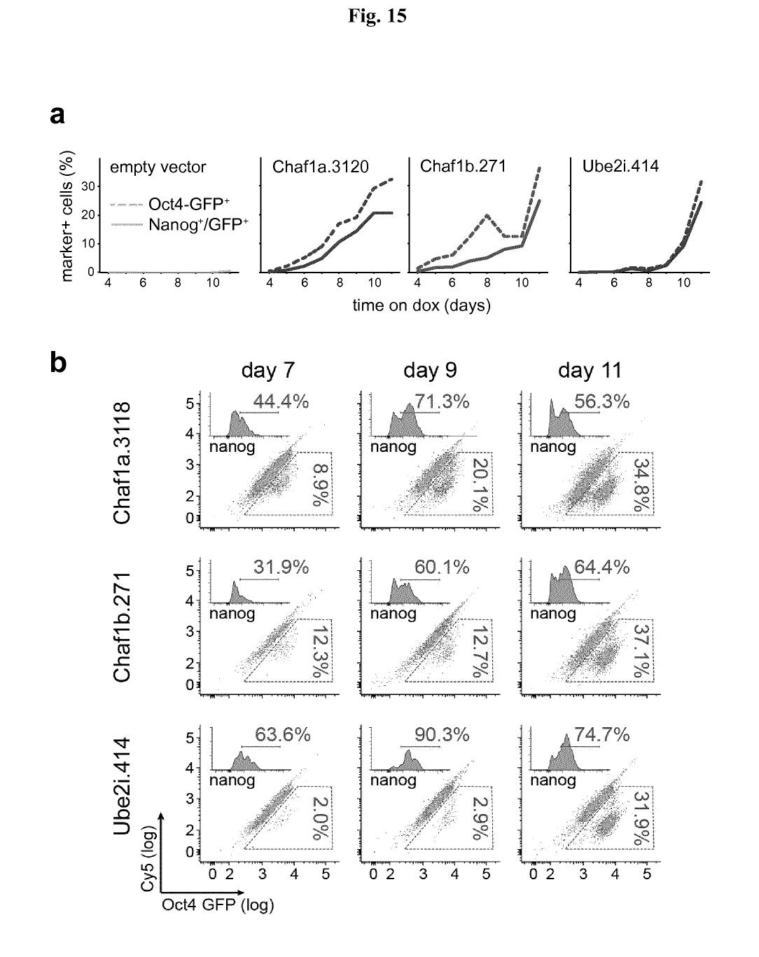

FIG. 15: RNAi-mediated suppression of Chaf1a, Chaf1b or Ube2i strongly enhance the formation of Nanog+ iPS cells. (a) Flow cytometry analysis of Oct4-GFP reporter expression and intracellular flow cytometry for Nanog expression during the reprogramming of reprogrammable MEFs harboring indicated shRNA vectors. (b) Representative FACS plots showing effects of Chaf1a/b or Ube2i knockdown on emergence of Oct4-GFP⁺ cells at days 7, 9, and 11 of OKSM expression. Histograms show fraction of Nanog⁺ cells within Oct4-GFP⁺ cells.

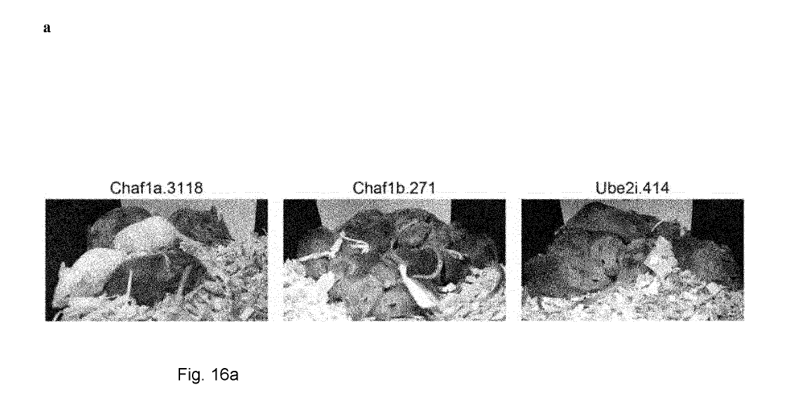

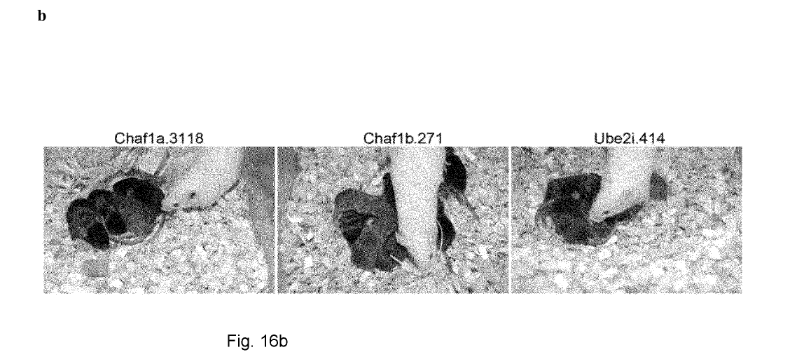

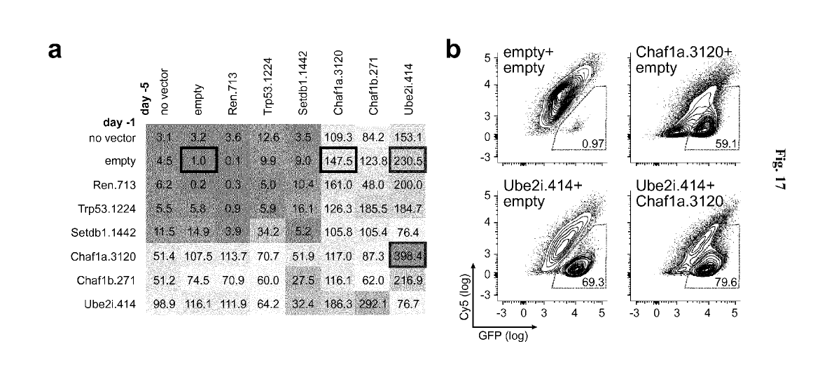

FIG. 16: Enhanced reprogramming through suppression of Chaf1a, Chaf1b or Ube2i yields developmentally fully competent iPS cells (a) Multiple high grade chimeras produced by injection of iPSCs (agouti pigment) into blastocysts (albino). iPSCs were obtained from reprogrammable MEFs after seven days of dox induction (OKSM expression) and concomitant shRNA expression of either Chaf1a, Chaf1b or Ube2i. Note that no iPSCs were recovered from control shRNAtreated cells within the same time period. Chimerism ranged from 95-100% for Chaf1a shRNA-derived iPSCs (3 mice), 80-100% for Chaf1b shRNA-derived iPSCs (12 mice), and 85-100% for Ube2i shRNA-derived iPSCs (8 mice). Albino mice represent nonchimeric littermates. (b) Germline transmission of agouti chimeras (Tyr % A/a) generated from iPSCs upon dox-induced knockdown of Chaf1a, Chaf1b, or Ube2i. Germline transmission was determined by agouti coat color contribution to offspring when chimeras were bred to albino females (Tyr⁺/Tyr^{c}; A/a and a/a as opposed to Tyr^{c}/Tyr^{c}; A/a and a/a). Germline transmission was observed in 8/8, 4/4, 6/8 cases for Chaf1a chimeras, in 7/7, 4/4, 7/7, 9/9 cases for Chaf1b chimeras, and in 5/5, 7/7, and 5/5 cases for Ube2i chimeras.

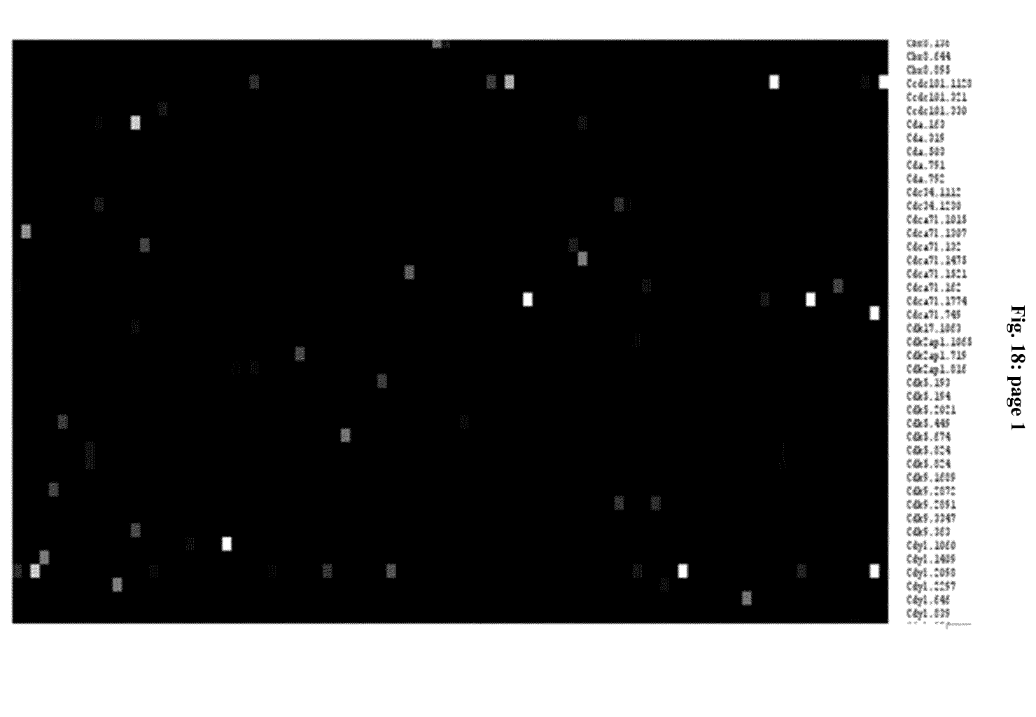

FIG. 17: Systematic combinatorial RNAi studies to explore synergistic RNAi effects in iPS reprogramming. (a) Table summarizing consequences of co-suppression of pairs of shRNAs on emergence of Oct4-GFP+ cells, shown as fold change relative to control (iPSCs/fibroblasts normalized to empty vector). (b) Representative flow cytometry plots of samples shown in (a). Combined suppression of CAF-1 components and Ube2i cooperate to enhance iPS reprogramming efficiency.

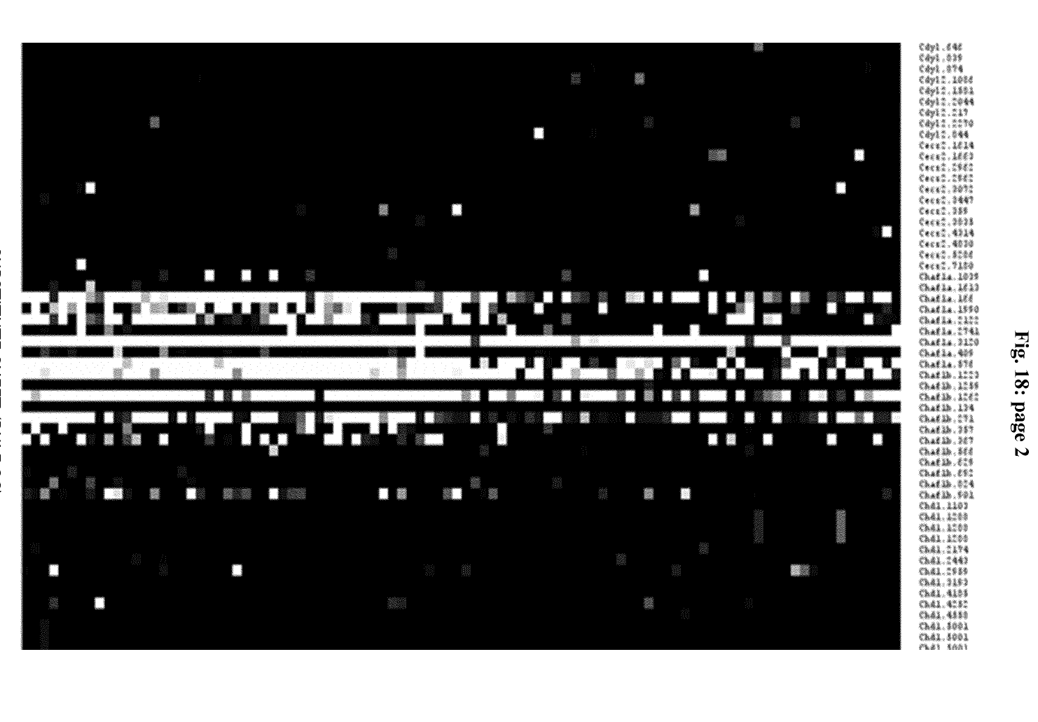

FIG. 18: Heatmap representation of the primary screening data. Shown is the relative enrichment of individual shRNAs in a continuous scale from black (no enrichment or depletion) to white (>30 fold enrichment), which was calculated based on the ratio between deep-sequencing reads in iPS cells in each replicate and the reads in infected MEFs prior to reprogramming. Columns represent all 96 screens (48 biological replicates, each run using two time points of OSKM induction). Rows represent individual shRNAs (5049 total, in alphabetical order); shown is a caption at the level of Chaf1/Chaf1b shRNAs. Multiple Chaf1a and Chaf1b shRNAs strongly enrich consistently between replicates, and clearly represent the top score of the screen.

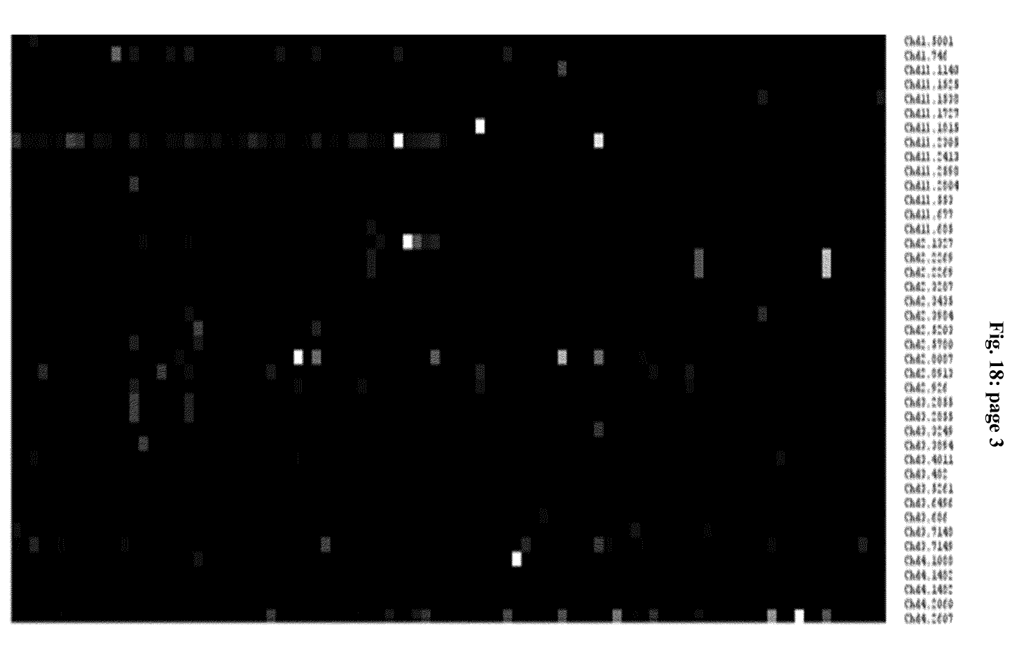

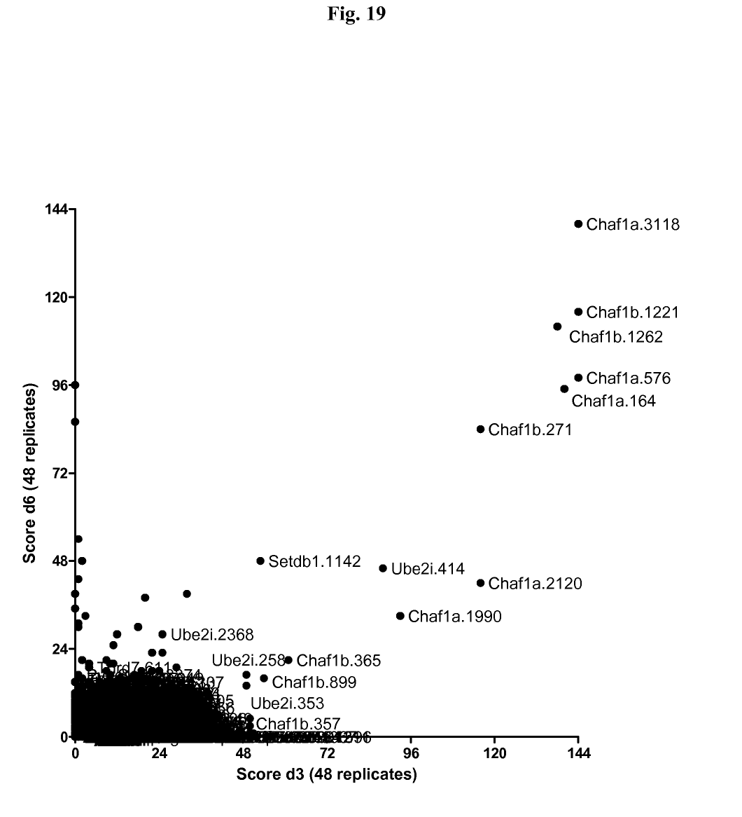

FIG. 19: Scatter plot representing the sum score of 5049 shRNAs across all replicates. In all 96 iPS samples (48 biological replicates, 3 or 6 days KD prior to OKSM) the normalized reads of each shRNA were divided by the normalized reads in MEFs 3 d after viral transduction, and the resulting ratio was used to calculate a score for each shRNA in each replicate (default score=0; score=1 if ratio>1, score=3 if ratio>10). Scores of each shRNA in 48 replicates were added separately for the d3 and d6 time point, yielding a sum score to estimate the overall enrichment of each shRNA over all replicates. Strongly scoring shRNAs targeting Chaf1a, Chaf1b, Ube2i and Setdb1 are annotated. Chaf1a, Chaf1b and Ube2i emerge as very clear top hits of the screen supported by multiple shRNAs.

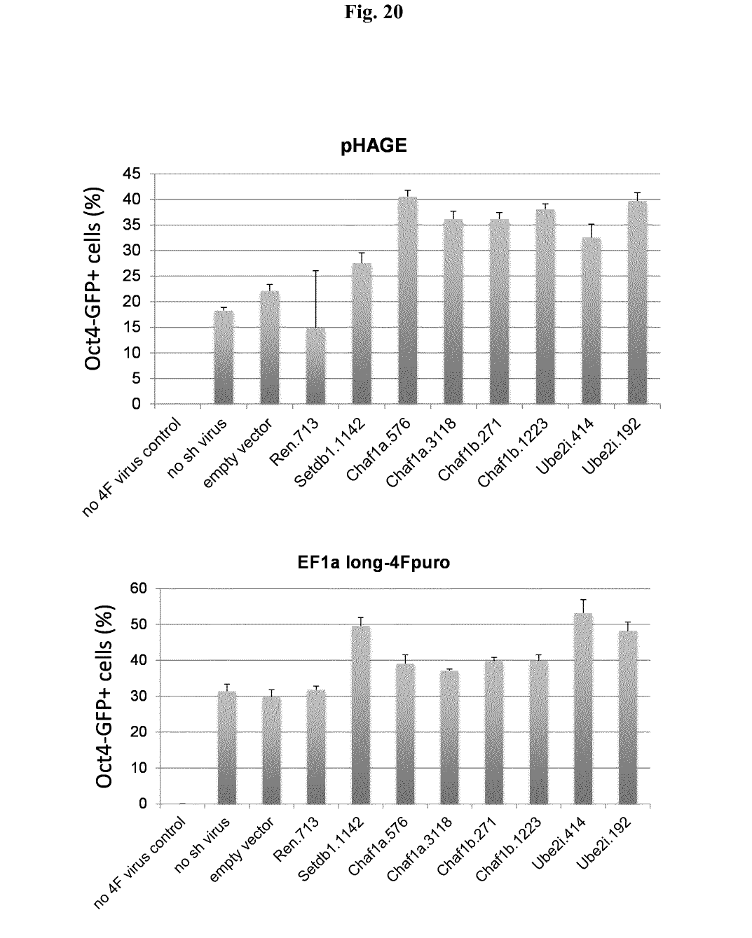

FIG. 20: Validation of enhanced reprogramming through Chaf1a, Chaf1b or Ube2i suppression using lentiviral vectors constitutively expressing OSKM. Oct4-GFP transgenic MEFs were first transduced with indicated pLENC-shRNAs, and subsequently (3 days later) transduced with lentiviral vectors constitutively expressing OKSM from a strong EF1-alpha promoter (pHAGE or EF1along-4Fpuro). Shown is the percentage of Oct4-GFP positive cells at day 11 after lentiviral infection. Error bars indicate standard deviation (SD) of 3 biological replicates. Suppression of Chaf1a, Chaf1b and Ube2i enhance iPS reprogramming. Due to a higher baseline reprogramming efficacy using lentiviral OSKM expression, the fold enhancement is lower compared to the primary system (ColA-TRE-OKSM/rtTA-M2/Oct4-GFP triple transgenic reprogrammable MEFs), suggesting that the effects of Chaf1a, Chaf1b and Ube2i expression are particularly potent under conditions were the expression or supply of OKSM factors is limited.

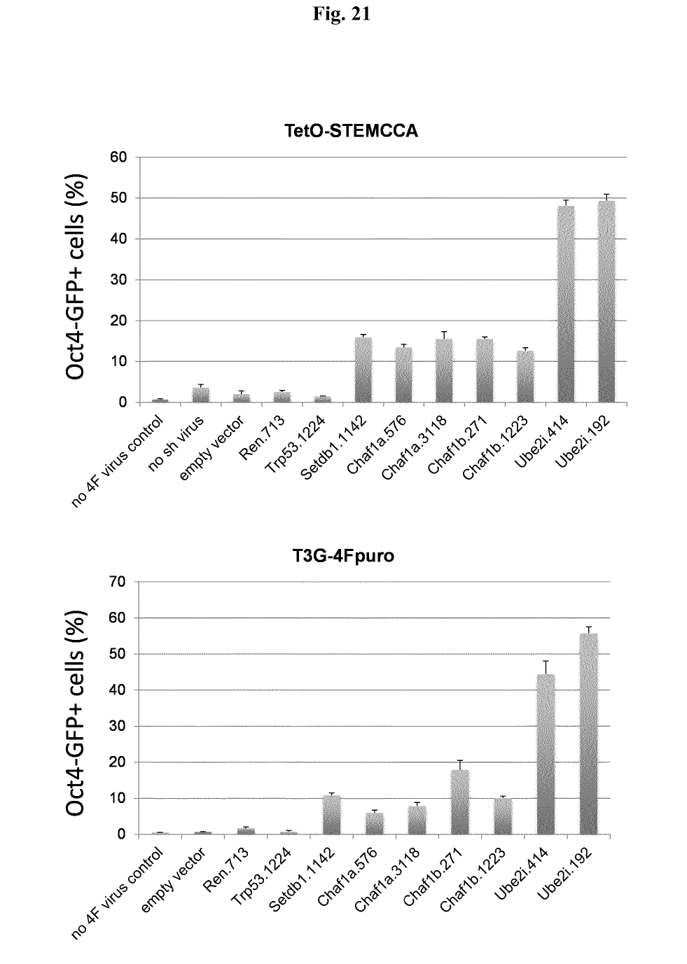

FIG. 21: Validation of enhanced reprogramming through Chaf1a, Chaf1b or Ube2i suppression using lentiviral vectors expressing Tet-inducible OSKM. Oct4-GFP;CAGGS-rtTA3 double-transgenic MEFs were first transduced with indicated pLENC-shRNAs, and 3 days later transduced with lentiviral vectors inducibly expressing OKSM from the Tet-responsive promoters TRE (TetO-STEMCCA) or TRE3G (T3G-4Fpuro) in the presence of doxycycline. To induce OSKM expression, cells were treated with doxycyline for a total of 7 days. Shown is the percentage of Oct4-GFP positive cells at day 11 after lentiviral infection. Error bars indicate standard deviation (SD) of 3 biological replicates. Suppression of Chaf1a, Chaf1b and Ube2i strongly enhance iPSC reprogramming using both Tet-inducible OKSM expression vectors. Therefore, improvement of reprogramming is not specific to the secondary system used in the primary screen.

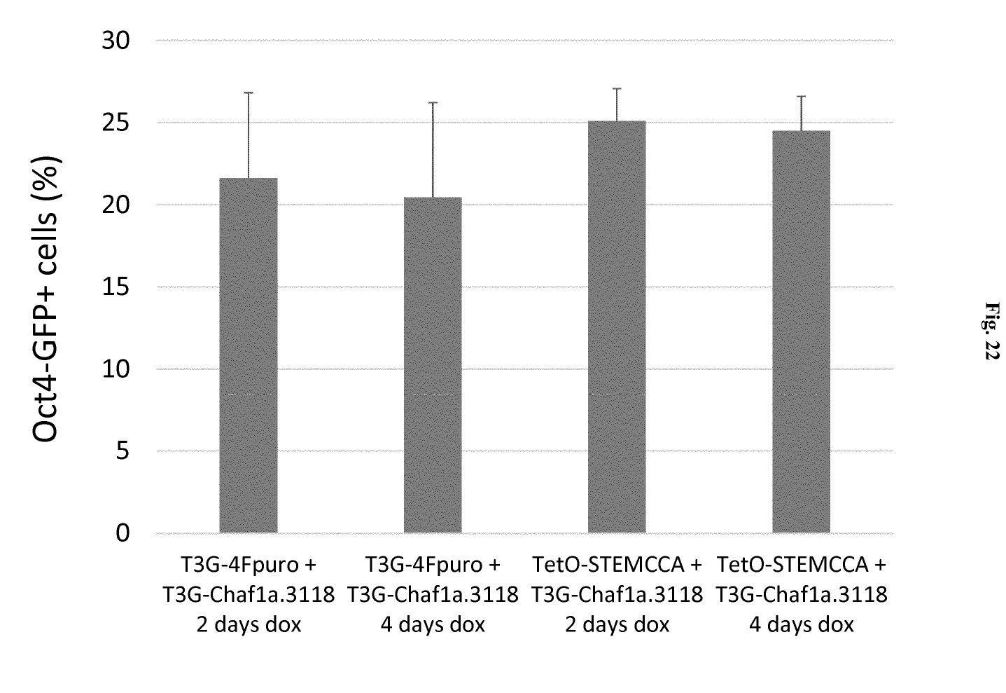

FIG. 22: Short-term transient expression of OKSM and suppression of Chaf1a for only 2-4 days enable robust iPSC reprogramming. Oct4-GFP;CAGGS-rtTA3 double-transgenic MEFs were first transduced with a Tet-regulatable shRNA expression vector encoding a potent Chaf1a shRNA (T3G-mCherry-miRE.Chaf1a.3118), and 3 days later transduced with lentiviral vectors inducibly expressing OKSM from the Tet-responsive promoters TRE (TetO-STEMCCA) or TRE3G (T3G-4Fpuro). Cell were treated with doxycyline for 2 or 4 days starting at day in OKSM infection, triggering a transient and reversible OKSM expression and Chaf1a suppression. Shown is the percentage of Oct4-GFP positive cells at day 11 after lentiviral infection. Error bars indicate standard deviation (SD) of 3 biological replicates. Suppression of Chaf1a, Chaf1b and Ube2i enhance iPSC reprogramming in the use of both Tet-inducible OKSM expression vectors. Transient OKSM expression in concert with transient suppression of Chaf1a for as little as 2 days leads to robust formation of iPS cells

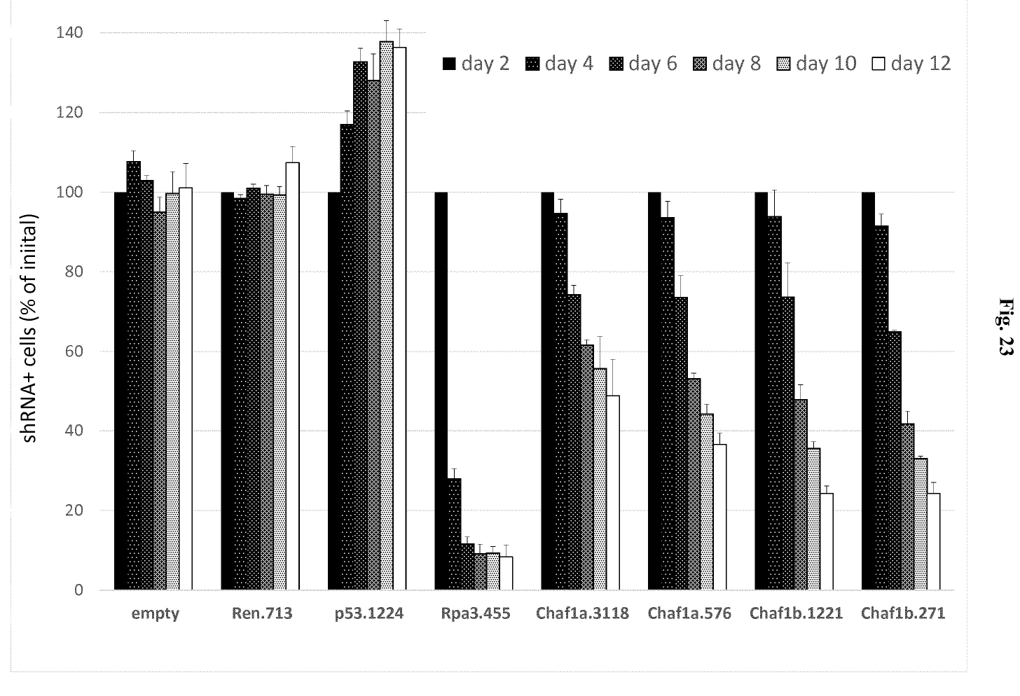

FIG. 23: Competitive proliferation assays to assay lethal effects of Chaf1a/b suppression in fibroblasts. NIH3T3 fibroblasts were transduced under single-copy conditions with pLENC vectors expressing the indicated top-scoring Chaf1a/b shRNAs and several control shRNAs. The percent of shRNA+ (mCherry+) cells was followed over time. For each shRNA bar graphs represent measurements 2, 4, 6, 8, 10 and 12 days following transduction. Similar to an shRNA targeting the DNA replication factor Rpa3, Chaf1a and Chaf1b suppression results in a rapid depletion of shRNA+ cells, indicating that Chaf1a/b suppression under conditions used in our screen is detrimental in fibroblasts. For the use of Chaf1a/b shRNAs for enhancing iPSC reprogramming, this suggests that transient Chaf1a/b suppression should be preferred over stable Chaf1a/b suppression.

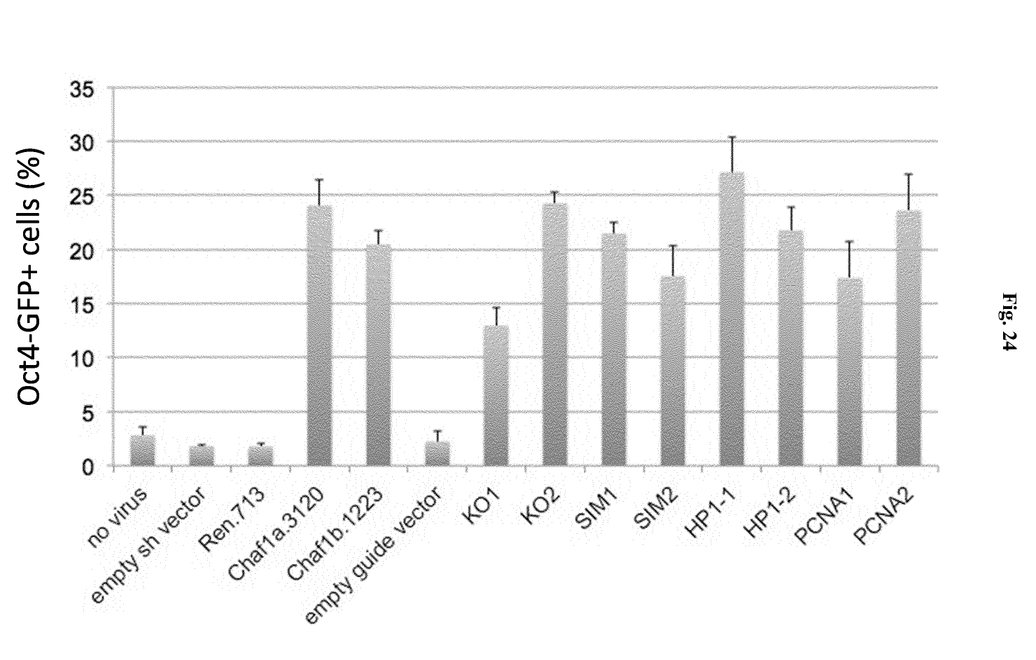

FIG. 24: Validation of enhanced reprogramming through genetic Chaf1a disruption. In order to test, if stable disruption of Chaf1a alleles can improve reprogramming, we targeted Chaf1a in OKSM inducible MEFs using CRISPR Cas9 technology. To this end, we designed guide RNAs and delivered them via lentivirus into MEFs. 2 days post infection, MEF cells were sorted by FACS for successful transduction making use of a Thy1.1 surface antigen as reporter. Guides were designed to disrupt Chaf1a by mutagenesis of various domains such as between protein domains (KO1 and 2), within the sumo interaction domain (SIM1 and 2), within the HP1 interaction domain (HP1 and 2), as well as within the PCNA binding domain (PCNA1 and 2). Genome editing was allowed to proceed for 7 days. At day 7, equal number of MEFs were plated in biological triplicate and the OKSM transgene was induced by doxycycline for 7 days. IPS formation was scored on day 11 post induction by FACS making use of the Oct4 driven GFP reporter system as previously described. FIG. 24 shows reprogramming efficiency to be comparable to sh mediated knockdown when targeting across the Chaf1a coding region. Therefore, enhanced reprogramming is not dependent on RNAi mediated inhibition of the CAF1 complex and expected to occur upon inhibition of variable protein domains. This experiment strongly suggests, that pharmacological inhibition of CAF1 will also induce enhanced reprogramming.

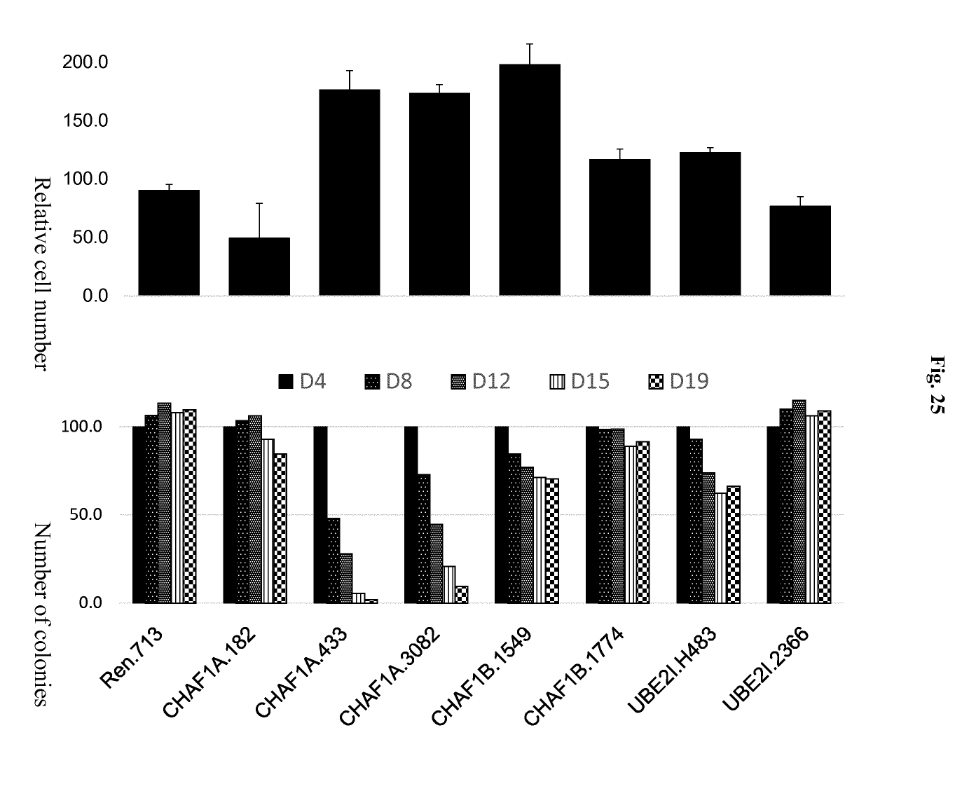

FIG. 25: Dermal fibroblasts were transfected with lentivirus to induce knockdown. subsequently, equal numbers of cells were plated and EF1alpha long promoter driven OKSM was used to induce reprogramming and colonies were counted visually based on alkaline phosphatase staining. Hairpins showing the best iPS colony formation (top) did so despite the fact that these hairpins display expected growth disadvantage in competition assay in human dermal fibroblasts (bottom).

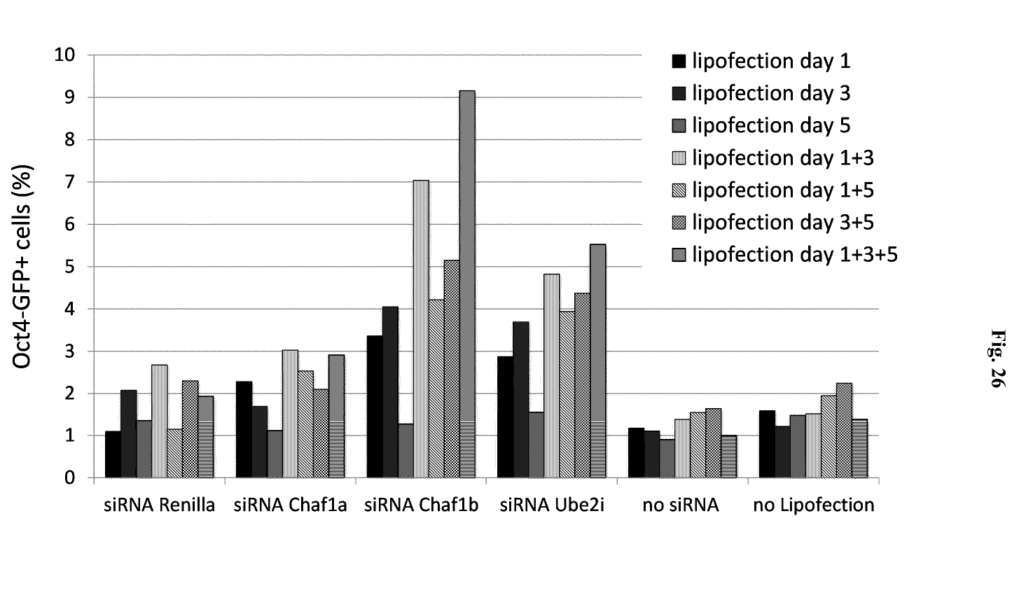

FIG. 26: Transient suppression of Chaf1a, Chaf1b or Ube2i using siRNA transfection enhances iPSC reprogramming. Reprogrammable transgenic MEFs (Tet-OKSM, Rosa26-rtTAM2, Oct4-GFP) where transfected 1 to 3 times at the indicated days of doxycyline treatment with siRNAs targeting Renilla, Chaf1a, Chaf1b, Ube2i. Shown is the fraction of Oct4-GFP positive cells after 11 days. Transient siRNA-mediated suppression of Chaf1a, Chaf1b or Ube2i enhances reprogramming. Suppression of these targets at early time points of reprogramming (day 1-3 following OKSM expression) seems critical and sequential transfections at different days can enhance this effect. These results also suggest that the degree and duration of CAF1 and Ube2i suppression are critical parameters that should be optimized. The siRNA molecules used in the study have the following sequence: Ube2i.414—2: CACAATTTACTGCCAAAACAA (SEQ ID NO:103), Chaf1a.3120 CAGCTACTTCCAAATTGTAAA (SEQ ID NO:104), Chaf1b.271 TGGAATTTCTCTCCAATCTTA (SEQ ID NO:105) and Ren.713 AGGAATTATAATGCTTATCTA (SEQ ID NO:106).

### EXAMPLE 1: ENHANCED REPROGRAMMING TO iPS CELLS BY RELEASE OF EPIGENETIC ROADBLOCKS

Somatic cells can be reverted back to an embryonic stem cell like state by expression of a set of 4 transcription factors (Oct-4, Sox-2, Klf-4, c-Myc; so-called OSKM or Yamanaka factors). However, this process is extremely inefficient and stochastic with only very few cells reaching the induced pluripotent stem cell (iPS) state. It is generally believed that epigenetic memory of the initial state represents a major roadblock preventing efficient iPS reprogramming. To systematically explore factors involved in establishing epigenetic roadblocks of iPS reprogramming, we have screened a new customized shRNAmir library (5100 shRNAs targeting 650 known and predicted chromatin regulators) in an established reprogramming assay. We identify and validate several novel factors whose suppression leads to a dramatic increase in iPS reprogramming efficacy, demonstrating that the release of epigenetic roadblocks can turn this stochastic and inefficient process into a highly effective method. Based on the quality of our reagents and the systematic nature of our approach, we believe the identified genes represent the most potent chromatin-associated targets for releasing epigenetic roadblocks in iPS reprogramming and, potentially, other cell-fate conversion methods. Moreover, in contrast to previous reprogramming factors, several of these genes are amenable to pharmacologic modulation and therefore represent a first set of targets for chemically induced cellular reprogramming Inhibiting these factors using RNAi, other genetic or small-molecule approaches will establish highly efficient and, potentially, simplified and safer iPS reprogramming regimens, which will be of great utility for a broad spectrum of basic research and biomedical applications.

### Explanation of Research and Data

Since its discovery, cellular reprogramming to pluripotency has become a broadly used experimental tool. Beyond its great utility in basic and biomedical research, iPS reprogramming is believed to be applicable for a wide range of medical applications such as the generation of patient-specific tissue for cellular therapy. However, the process of iPS reprogramming remains very inefficient and stochastic in nature, which diminishes its utility for many applications, particularly if the source of somatic cells is limited. While the major roadblock preventing efficient iPS reprogramming is thought to lie in the hard-wired epigenetic landscape, the key mechanisms and factors contributing to this roadblock remain incompletely understood [1,2].

To systematically identify chromatin-associated factors involved in preventing iPS reprogramming we sought to perform a functional genetic screen that takes advantage of established reprogramming assays in mouse embryonic fibroblasts (MEFs) and a novel shRNAmir library targeting all known and predicted chromatin regulators (650 genes, ˜5,100 sequence-verified shRNAs in an equimolar pool). By incorporating Sensor-based predictions and the pLENC vector featuring the improved miR-E backbone [3], this library (unlike previous miR30-based and other available RNAi reagents) contains a majority of shRNA constructs that trigger potent (>80%) protein knockdown under single-copy conditions, which for the first time enables a truly systematic analysis of the entire chromatin network in a multiplexed format.

As cellular screening model, we chose to use previously established MEFs engineered to carry: (1) an expression cassette harboring the 4 OSKM factors under control of a Tet-responsive element (TRE), (2) the reverse Tet-transactivator rtTA-M2 driven from the Rosa26 promoter (RR), and (3) an Oct4 promoter-driven GFP transgene (OG) for identification of iPS cells [4], which were established and provided by the lab of Konrad Hochedlinger (MGH/Harvard). Transgenic OSKM/RR/OG-MEFs can be reprogrammed at low efficacy through addition of doxycyline (Dox) to the culture media, and therefore provide a controllable and reproducible iPS reprogramming model, which is ideally suited for multiplexed screening.

To conduct the screen in a multiplexed/pooled format and reduce biases due to sporadic reprogramming events, we devised the following screening strategy: OSKM/RR/OG-MEFs isolated from 4 independent embryos were grown at low oxygen in the presence of ascorbate [5], retrovirally transduced with a pool of 5,100 pLENC-shRNA vectors (co-expressing miRE-based shRNAs, mCherry and a Neo resistance gene) and selected with G418. OSKM expression was induced using Dox treatment for 7 days starting either 3 or 6 days after transduction of the library. For both conditions the screen was performed in 48 independent biological replicates (96 total) each containing an >100-fold representation of the library, which were handeled separately throughout the entire experiment. Plates were trypsinized 11 days after OSKM induction, MEFs were reduced by pre-plating, and remaining cells were reseeded on equal surface.

On day 18 after OSKM induction, from each replicate 3-5 million iPS cells were FAC-sorted based on GFP expression and cell size, genomic DNA was extracted, shRNA guide strands were amplified (using an optimized PCR protocol that directly tags Illumina adaptors and sample barcodes), PCR products were subjected to deep sequencing, and deep sequencing data were analyzed using a customized Galaxy workflow. The representation of each shRNA in the library was quantified in all 96 samples, and compared to the representation in OSKM/RR/OG-MEFs 3 days after library transduction. Overrepresented shRNAs were identified in each sample, individual samples were integrated to an overall shRNA score, which then was integrated to a gene score that takes into account the number of shRNA, the number of scoring replicates and the scale of the effect. Notably, for some of the top scoring genes several independent shRNAs enriched very strongly and consistently throughout the large number of replicates. Table 1 shows the list of identified candidate genes.

TABLE 1
Top 24 screen hits in the primary screen ranked by a score that
takes into account the number of scoring shRNAs, the number of
independent scoring replicates and the severity of the effects.
Provide are mouse gene symbols, the total shRNAs in the library,
the number of scoring shRNAs, the fold-change enrichment of the
top scoring shRNA for each gene (FC max) and the score.

| | | shRNAs | shRNAs | | |
|---|---|---|---|---|---|
| Rank | Gene | total | scoring | FC max | Score |
| 1 | Chaf1a | 9 | 8 | 452.7 | 153958.3 |
| 2 | Chaf1b | 12 | 6 | 164.3 | 24810.8 |
| 3 | Ube2i | 12 | 8 | 24.1 | 2447.7 |
| 4 | Setdb1 | 11 | 3 | 10.5 | 236.2 |
| 5 | Ube2a | 8 | 1 | 49.8 | 227.4 |
| 6 | Mbd4 | 12 | 1 | 39.4 | 167.3 |
| 7 | Chd4 | 12 | 4 | 18.5 | 151.6 |
| 8 | Setd2 | 17 | 4 | 11.1 | 150.8 |
| 9 | Meaf6 | 4 | 2 | 10.2 | 109.6 |
| 10 | Hdgfl1 | 5 | 3 | 9.1 | 92.6 |
| 11 | Ubr4 | 8 | 3 | 6.2 | 87.2 |
| 12 | Men1 | 13 | 3 | 11.3 | 86.9 |
| 13 | Rnf2 | 12 | 2 | 14.5 | 70.3 |
| 14 | Brdt | 11 | 3 | 21.0 | 69.0 |
| 15 | Brd4 | 13 | 4 | 6.8 | 65.5 |
| 16 | Nono | 4 | 2 | 4.0 | 49.5 |
| 17 | Csnk2a1 | 8 | 2 | 7.6 | 49.1 |
| 18 | Uhrf1 | 7 | 3 | 11.4 | 43.3 |
| 19 | Atm | 16 | 3 | 36.2 | 43.0 |
| 20 | Atrx | 10 | 3 | 4.0 | 48.4 |
| 21 | Baz1b | 10 | 3 | 18.6 | 41.8 |
| 22 | Atr | 15 | 2 | 17.2 | 37.2 |
| 23 | Mll5 | 16 | 3 | 7.3 | 31.5 |
| 24 | Daxx | 5 | 1 | 7.1 | 28.2 |

To validate hits identified in the primary multiplexed screen, a set of 32 independent shRNAs including several of the top hits as well as a panel of intermediately scoring shRNAs were tested in single assays. To this end, OSKM/RR/OG-MEFs were transduced with individual pLENC shRNAs in biological triplicates, selected, and treated with Dox for OSKM induction. The iPS reprogramming efficacy was quantified using flow cytometry analysis of the Oct-4-GFP reporter (FIG. 1) and microscopy (FIG. 2a).

Compared to controls (Ren.713, no shRNA) most tested shRNAs lead to a marked increase in reprogramming efficiency, many in the range of an shRNA targeting Trp53 (previously implicated as a reprogramming roadblock), suggesting that the multiplexed screen successfully identified genes that prevent iPS cell formation. The most dramatic increase in reprogramming efficacy was observed for 4 genes: Chaf1a, Chaf1b, Setdb1 and Ube2i, which also represent the 4 top hits in our analysis of the multiplexed screen (Table 1). RNAi-mediated suppression of these genes lead to 20%-50% Oct4-GFP+ cells (compared to <1% in controls; FIG. 1) and a striking boost in iPS colony formation (FIG. 2). The degree of these effects exceeds by far the increase observed for previously established roadblock factors, and is only matched by a heavily questioned paper describing MBD3 as such factor (which we and others cannot reproduce), and recent reports about stimulus-triggered acquisition of pluripotency (STAP), which are about to be retracted.

While Chaf1a, Chaf1b and Ube2i have not been previously implicated as reprogramming roadblocks, Setdb1 (the 4^{th} strongest of our hits) has recently been described as a barrier to iPS formation [6], and thus serves as a perfect internal control to the experimental setup. Two of the three previously unknown top hits (Chaf1a and Chaf1b) are known to form a complex called CAF1, which is required for loading of core histones onto nascent DNA during S-phase. Based on this established function we hypothesize that inhibition of CAF1 interferes with the maintenance of epigenetic memory during cell division, which ultimately renders cells more susceptible to cell fate conversions such as iPS reprogramming.

The 3^{rd} top hit in our multiplexed screen is the SUMO E2 conjugating enzyme Ube2i/Ubc9, whose knockdown caused the single most potent effect in our primary validation study (FIG. 1). As Ube2i is the only E2 ligase, it has to be assumed that SUMOylation is globally defective following Ube2i knockdown, suggesting that SUMOylation represents a genetic roadblock to iPS reprogramming through a yet unknown downstream target/mechanism.

Importantly, both newly identified pathways (i.e. histone loading by Chaf1a/b and SUMOylation) involve enzymatic activities that unlike OSKM factors and most previously described roadblocks are amenable to drug interference. Possible substances to suppress these pathways and thereby enhance iPS reprogramming might include roscovitine [7] for CAF-1, as well as H₂O₂, spectomycin B1, chaetochromin A, viomellein, and davidiin for UBE2I [8]. Additionally, SUMOylation may be inhibited at other levels of the reaction cascade such as by E1 inhibitors [9] or elsewhere.

Of note, beyond the 4 top hits several other scoring and validating shRNAs target factors for which pharmacologic inhibitors are already available. Examples include BrdT and Brd4, for which several validated shRNAs of intermediate knockdown potency lead to a clear enhancement in reprogramming efficacy. Another example is Csnk2a1 (casein kinase 2, alpha 1 polypeptide), for which potent inhibitors such as CX-4945/Silmitasertib already exist. Beyond testing these inhibitors individual for their potential to enhance reprogramming, it is conceivable that the systematic functional genetic data generated in our study could be used to design compound regimens (i.e. a “reprogramming cocktail”) for enhancing the efficacy of iPS cell. Whether the drug- or RNAi-mediated suppression of one or several roadblock factors can be used to establish simplified iPS reprogramming protocols (e.g. without Myc or other OSKM factors) or facilitate direct tissue reprogramming will need to be determined in follow-up studies.
[1] Apostolou, E. and Hochedlinger, K. (2013). Chromatin dynamics during cellular reprogramming. Nature 502, 462-71.
[2] Orkin, S. H. and Hochedlinger, K. (2011). Chromatin connections to pluripotency and cellular reprogramming. Cell 145, 835-50.
[3] Fellmann, C. et al. (2013). An optimized microRNA backbone for effective single-copy RNAi. Cell Rep 5, 1704-13.
[4] Stadtfeld, M., Maherali, N., Borkent, M. and Hochedlinger, K. (2010). A reprogrammable mouse strain from gene-targeted embryonic stem cells. Nat Methods 7, 53-5.
[5] Stadtfeld, M. et al. (2012). Ascorbic acid prevents loss of Dlk1-Dio3 imprinting and facilitates generation of all-iPS cell mice from terminally differentiated B cells. Nat Genet 44, 398-405, S1-2.
[6] Chen, J. et al. (2013). H3K9 methylation is a barrier during somatic cell reprogramming into iPSCs. Nat Genet 45, 34-42.
[7] Keller, C. and Krude, T. (2000). Requirement of Cyclin/Cdk2 and protein phosphatase 1 activity for chromatin assembly factor 1-dependent chromatin assembly during DNA synthesis. J Biol Chem 275, 35512-21.
[8] Hirohama, M. et al. (2013). Spectomycin B1 as a novel SUMOylation inhibitor that directly binds to SUMO E2. ACS Chem Biol 8, 2635-42.
[9] Fukuda, I. et al. (2009). Ginkgolic acid inhibits protein SUMOylation by blocking formation of the E1-SUMO intermediate. Chem Biol 16, 133-40.

### EXAMPLE 2: FURTHER INFORMATION AND DATA ON THE INVENTION

The initial screen as well as validations of primary hits was done in presence of ascorbate shown to facilitate iPS cell formation. Ascorbate is thought to reduce repressive histone modifications[1]. Furthermore, all experiments were done in ESC medium containing 15% FCS (Invitrogen) and 2×LIF. FCS was previously shown to have inhibitory effects on reprogramming due to Bmp-4 presence [1]. Setdb1 is presented in the cited publication as a required factor for the inhibitory effect of FCS since knockdown of Setdb1 releases the FCS triggered block. Additional experiments (below) have now tested the enhanced reprogramming regime identified by us in various settings thereby broadening the described finding.

IPS formation generally is thought to be a process, whereby differentiated cells, more specifically mouse embryonic fibroblasts (MEFs) in our case, first transit through a stage termed pre-iPS cells [1]. Pre-iPS cells are identified as cells with partially iPS cell identity. Thus, in principle, iPS cell formation from MEFs can be improved by either enhancing pre-iPS formation or the final cell identity switch from pre-iPS cells to iPS cells[2]. Herein presented invention claims to deliver evidence for improvement of either step to enhance the final reprogramming outcome. While Caf1 complex inhibition appears to enhance pre-iPS cell formation, inhibition of Ube2i removes the roadblock of the pre-iPS to iPS transition.

The first validation experiment showed a dramatic improvement of iPS formation with several hairpins. In order to exclude that this effect is limited to a particular timing, we tested induction of OSKM 3 days (D3) and 6 days (D6) after transduction of hairpins. Very similar effects were noticed. As expected, reprogramming is generally slightly less efficient on D6 due to the increased passaging time of primary MEFs. Notably, knockdown of Caf-1 proteins Chaf1a and Chaf1b did not result in the expected reduction arguing for Caf-1 loss to be essential at the onset of reprogramming, where full loss might not have been established on D3 just 72 h after transduction. (see Table 1 in Example 1).

### A. Timeline of Oct4-GFP Expression During Reprogramming

### 1) FACS Based Timeline Analysis

In order to observe the kinetics of reprogramming in the enhanced reprogramming regimen, we quantified GFP positive cells using FACS analysis. GFP driven from the endogenous Oct4 locus serves as a marker for Oct4 expression and thus iPS and possibly pre-iPS state. As can be seen from FIG. 3, suppression of Caf-1 markedly accelerated reprogramming but leads to a steady state around day 10. In contrast, Ube2i suppression only slightly accelerated the reprogramming timeline, but made the process much more efficient, and ultimately exceeds the effects of observed for Caf-1 suppression (i.e. the percentage of GFP+ cells actually overtakes the enhancement achieve through Caf-1 suppression at day 10).

This may be due to a negative effect of Caf-1 suppression at later stages of iPS reprogramming and/or by a preferential effect of Caf-1 suppression during early reprogramming steps such as the formation of pre-iPS cells. In case Caf-1 suppression has inhibitory effects during late stages of iPS formation and/or in the maintenance of established iPS, such detrimental effects could be reduced or avoided using methods that trigger only transient Caf-1 suppression (e.g. inducible shRNA expression, siRNA/shRNA transfection, transient drug treatment), which may have the potential to further enhance iPS reprogramming efficacy.

### 2) Endpoint Analysis Upon Split and DOX Withdrawal

Next, we sought to determine the time point at which cells become independent of ectopic OSKM expression and gain the ability to form iPS colonies, indicative of complete dedifferentiation to iPS cells and establishment of the feed-forward loop of stem cell circuitry. To this end, we split iPS cells at various time points and removed doxycycline. In sharp contrast to the timeline in FIG. 3, enhancement of reprogramming provided by Caf-1 and Ube2i suppression follows very similar kinetics. Most fully dedifferentiated iPS cells are born between day 6 and 8 in all cases of enhanced reprogramming, while control iPS cells require >10 days to establish. Therefore, the highly abundant GFP+ cells observed under Chaf1a/b suppression (FIG. 3) most likely represent pre-iPS cells, while Ube2i suppression leads to iPS formation in a much higher fraction of cells.

### B. Testing Enhanced Reprogramming in Various Conditions

As mentioned previously, additional experiments were designed to test the improved reprogramming regimen in different contexts.

To this end, described MEFs with doxycycline inducible reprogramming factors (OSKM, 4F, Yamanaka factors) were transduced with pLENC-shRNA vectors (co-expressing miRE-based shRNAs, mCherry and a Neo resistance gene) in biological triplicate as described previously. Selection for infected cells was based on G418 resistance. 3 days after infection, cells were trypsinized and each replicate was plated in multiple wells in presence of FCS and ascorbate containing medium as well as doxycycline. Cells were subjected to various conditions for iPS formation starting 24 h after Doxycycline addition, i.e. prior to expected full expression of OSKM factors. Doxycycline was added to media for 7 days. Analysis of iPS cell formation was 11 days after addition of Doxycycline using FACS analysis based on the expression of GFP driven by the endogenous Oct4-promoter.

MEFs used for this experiments were prepared in house from e14.5 embryos. Females used for the breeding carried an rtTA cassette integrated into the ROSA locus as previously described but are in house bred. Thereby, the experiment further illustrates independence of our finding to particular mouse backgrounds.

1) Serum with Ascorbate

A repetition of the previous condition used for first validation was done to demonstrate reproducibility of the finding in independent MEFs and as an independent experiment as well as to set the basis for comparison with alternative regimen. See FIG. 5.

As previously, knockdown of identified reprogramming roadblocks leads to a dramatic improvement of reprogramming. In conclusion, the described effects are reproducible in quality.

2) Serum without Ascorbate

To rule out that release of reprogramming roadblocks depends on presence of ascorbate, we used a fraction of transduced MEFs to induce reprogramming in absence of ascorbate in otherwise identical medium conditions. As can be seen in FIG. 6, improved reprogramming shows to be independent of ascorbate, while control conditions show expected reduction in reprogramming efficiency.

Thereby, the factor of improvement of reprogramming efficiency in this set of experiments is even higher.

3) Serum Replacement without Ascorbate

As mentioned previously, fetal calf serum (FCS) contains Bmp-4 with inhibitory effects upon iPS cell formation. Furthermore, different FCS batches support growth and formation of iPS cells to varying degree. To test if the described release of the reprogramming roadblock can be recapitulated in serum replacement representing standardized conditions, we used the same genetic system also in these conditions. As shown in FIG. 7, as expected, iPS formation in control genetic conditions (e.g. neutral or no hairpin) is enhanced. Enhanced reprogramming with knockdown also shows improvement in serum replacement.

4) Serum Replacement without Ascorbate, with 2i, Colony Formation Assay

In order to test the achieved factor of iPS formation enhancement in a colony formation assay, we chose optimal conditions described for iPS formation, i.e. serum replacement, LIF and 2i. The two inhibitors (2i) are shown to support the ground state of pluripotency and enhance iPS formation. More specifically, 2i supports the transition of pre-iPS cells to iPS cells[3].

In doing so, we intended to measure the factor of improvement in optimal, potentially saturated conditions. 10000 cells were plated/10 cm dish and reprogramming was induced by addition of doxycycline for 7 days. Reprogramming in presence of 2i furthermore represents an additional condition to test versatility of herein described enhanced reprogramming regimen.

As discussed, inhibition of Caf-1 releases the roadblock towards pre-iPS formation. In presence of LIF and 2i, the transition towards iPS cell formation is no bottleneck in the doxycycline inducible OSKM system. Therefore iPS colony formation is dramatically improved upon Chaf1a or Chaf1b knockdown. Knockdown of Ube2i on the other hand, putatively relevant for the pre-iPS to iPS transition shows less effect in presence of 2i. However, observed colonies in Ube2i knockdown condition are reminiscent of uniform iPS colonies with no pre-iPS contribution (Table 2 and FIG. 8).

TABLE 2
number of colonies in serum replacement,
2i plus LIF at single cell density.

| Condition | Colony number |
|---|---|
| No vector | 1 |
| Empty vector | 2 |
| shRNA.Ren.713 | 0 |
| shRNA.Chaf1a | 422 |
| shRNA.Chaf1b. | 67 |
| shRNA.Setdb1. | 19 |
| shRNA.Ube2i | 46 |

### C. Transcriptional Effect of Enhanced Reprogramming on Doxycycline Inducible OSKM

In order to rule out that the improvement in iPS reprogramming is merely due to an enhancement in the ectopic expression levels of OSKM factors, we performed quantitative RT-PCR with a primer pair specific to the OSKM transgene and normalized expression levels to actin. Q-PCR was in triplicate on each of 3 biological triplicates.

As can be seen in FIG. 9, OSKM expression levels after suppressing the identified roadblock factors is within the same range and about 100-200 fold higher than in the uninduced condition (i.e. without doxycycline).

### D. Proposed Model and Future Experiments:

### Accelerating iPS Formation at Two Steps

Together, both timeline experiments and the phenotypic characterization of cells during reprogramming suggest that Caf-1 and Ube2i suppression enhance the formation of iPS cells at two different steps, potentially through independent mechanisms. Caf-1 suppression strongly promotes and accelerates the early steps of reprogramming, specifically the formation of pre-iPS cells, which is evidenced by the rapid and massive appearance of Oct4-GFP expressing cells. At later stages, Caf-1 suppression does not lead to a further enhancement, and many of the resulting colonies show only partial alkaline phosphatase staining, suggesting that continued Caf-1 suppression has no favourable or, potentially, even adverse effects on the transition from the pre-iPS to the iPS state. Therefore, strategies enabling a transient suppression of Caf-1 at early reprogramming stages while restoring its endogenous expression at the pre-iPS to iPS transition might further increase the efficacy of Caf-1 suppression in reprogramming. In contrast, Ube2i suppression does not accelerate the early steps of reprogramming, but leads to a massive increase in the formation of fully developed iPS colonies. According to this model, we predict that combining an initial and transient suppression of Caf-1 with inhibition of Ube2i (or other components of SUMO pathways) throughout or at later stages of the reprogramming process may further facilitate and increase the efficacy of iPS formation. See FIG. 10.

### E. Detailed Experimental Procedures:

shRNA Library Generation:

A chromatin-focused shRNA library targeting 650 genes was custom-designed based on improved Sensor predictions, cloned from on-chip synthesized oligos and sequence verified using an in-house pipeline. The shRNAmir pool used in the screen was produced by shuttling ˜5100 sequence-verified equimolarly pooled shRNAs into pLENC featuring the miR-E backbone [3].

Next-generation sequencing: Deep sequencing was performed on an Illumina 2500, and data was analyzed using a customized Galaxy workflow.

Cell culture: Standard cell culture techniques were used, iPS cell induction was performed as described in [4].

FACS: Cells were sorted on a BD Aria-III and gated for FSC, SSC, GFP, and Cherry.

### F. Materials Used:

### Plasmids

For the pooled RNAi screen, shRNAs were expressed from the pLENC vector [4]. For screen validation, mouse shRNAs were cloned individually into pLENC, which has been described previously [4].

### Cell Culture and Media

Packaging cells (Platinum-E Retroviral Packaging Cell Line) for producing retrovirual particles were cultured in DMEM supplemented with 15% FBS, 100 U ml⁻¹ penicillin, 100 μg ml⁻¹ streptomycin, 2 mM L-Glutamine, and sodium pyruvate (1 mM) at 37° C. with 5% CO2.

293FT cells for producing Lentivirus were cultured in DMEM supplemented with 15% FBS, 100 U ml⁻¹ penicillin, 100 μg ml⁻¹ streptomycin, 2 mM L-Glutamine, and sodium pyruvate (1 mM) at 37° C. with 5% CO2.

Mouse embryonic Fibroblasts were cultured in DMEM supplemented with 15% FBS, 100 U ml⁻¹ penicillin, 100 μg ml⁻¹ streptomycin, 2 mM L-Glutamine, sodium pyruvate (1 mM) 1×NEAA (non essential amino acids), 50 uM beta-Mercaptoethanol and L-ascorbic acid (50 uM) at 37° C. with lox oxygen (4.5% O₂). iPS cells were cultured in DMEM supplemented with 15% FBS, 100 U ml⁻¹ penicillin, 100 μg ml⁻¹ streptomycin, 2 mM L-Glutamine, sodium pyruvate (1 mM), 1×NEAA, 50 uM beta-Mercaptoethanol and 1000 U/ml LIF (i.e. ESGRO) at 37° C. with 5% CO2.

### ESC Media

For testing the influence of culture conditions of Reprogramming cells were cultured in following conditions during OSKM expression:
Reprogramming-standard ESC media: DMEM supplemented with 15% FBS, 100 U ml⁻¹ penicillin, 100 μg ml¹ streptomycin, 2 mM L-Glutamine, sodium pyruvate (1 mM), 1×NEAA, 1000 U/ml LIF and 50 uM beta-Mercaptoethanol.
Reprogramming-standard ESC media with L-ascorbic acid: DMEM supplemented with 15% FBS, 100 U ml⁻¹ penicillin, 100 μg ml⁻¹ streptomycin, 2 mM L-Glutamine, sodium pyruvate (1 mM), L-ascorbic acid 50 uM), 1×NEAA, 1000 U/ml LIF and 50 uM beta-Mercaptoethanol.
Reprogramming with Serum Replacement: DMEM supplemented with 13% Knockout Serum Replacement (i.e. Gibco), 2% FBS, 100 U ml⁻¹ penicillin, 100 μg ml⁻¹ streptomycin, 2 mM L-Glutamine, sodium pyruvate (1 mM), 1×NEAA, L-ascorbic acid (50 uM), 1000 U/ml LIF and 50 uM beta-Mercaptoethanol.
Reprogramming with Serum Replacement and 2i: DMEM supplemented with 13% Knockout Serum Replacement (e.g. Gibco), 2% FBS, 100 U ml⁻¹ penicillin, 100 μg ml⁻¹ streptomycin, 2 mM L-Glutamine, sodium pyruvate (1 mM), 1×NEAA L-ascorbic acid (50 uM), 1000 U/ml LIF, 50 uM beta-Mercaptoethanol, MEK inhibitor (1 μM) (i.e. StemMACS) and GSK3 inhibitor (3 μM) (i.e. StemMACS).

### Retroviral Transduction and Infection of Embryonic Mouse Fibroblasts (OSKM MEFs)

shRNAs were transduced into OSKM MEFs. After 36 h transduced cells were selected with 0.5 mg ml⁻¹ G418 for 3 days and 0.25 mg ml⁻¹ G418 for additional 3 days. 3 or 6 days after shRNA transduction infected cells were washed with PBS (1×) and trypsinized with Trypsin-EDTA (1×) and 20 000 cells were plated into a 6-well. OSKM expression was induced for 7 days and cells were cultured in DMEM supplemented with 15% FBS, 100 U ml⁻¹ penicillin, 100 μg ml⁻¹ streptomycin, sodium pyruvate (1 mM), 1000 U/ml LIF, beta-Mercaptoethanol and 1 μg ml⁻¹ Doxycyclin. After 7 days of OSKM expression cells were cultured for additional 4 days without doxycycline to withdraw 4 factors. Cells were analyzed using a FACS BD LSRFortessa.

MEFs: Transgenic OSKM/RR/OG-MEFs [4] were supplied by Sihem Cheloufi in the laboratory of Konrad Hochedlinger, Harvard, Boston, Mass., USA.
[1] Chen, J. et al. (2013). H3K9 methylation is a barrier during somatic cell reprogramming into iPSCs. Nat Genet 45, 34-42.
[2] Apostolou, E. and Hochedlinger, K. (2013). Chromatin dynamics during cellular reprogramming. Nature 502, 462-71.
[3] Theunissen, T. W., van Oosten, A. L., Castelo-Branco, G., Hall, J., Smith, A. and Silva, J. C. (2011). Nanog overcomes reprogramming barriers and induces pluripotency in minimal conditions. Curr Bio121, 65-71.
[4] Fellmann, C. et al. (2013). An optimized microRNA backbone for effective single-copy RNAi. Cell Rep 5, 1704-13.

### EXAMPLE 3: FURTHER VALIDATION OF THE RESULTS OF THE SCREEN

The following example provides further experimental data supporting the present invention.

The data contained in FIGS. 18 and 19 show the results of experiments performed by the inventors using a “multiplexed screen” approach. The inventors then decided to valiate these results by individually assessing the reprogramming efficiency of shRNA molecules. In this experiment, those individual shRNA molecules identified as having reprogramming efficiency (as shown in FIG. 11). Here it can be seen that 20 out of 26 tested shRNAs significantly enhanced reprogramming in validation experiments.

All tested shRNAs targeting Chaf1a, Chaf1b, or Ube2i robustly enhanced the fraction of Oct4-GFP⁺ cells to 20-60%, which is greatly higher than an shRNA targeting Trp53, a known repressor of iPSC formation. That reprogramming efficiency was not dependent on the specific culture conditions used in the experiment, as can be seen from FIG. 12. In addition, of Chaf1a, Chaf1b or Ube2i strongly increased the formation of dox-independent colonies that expressed AP and Nanog, indicating an effective acquisition of a transgene-independent iPSC-like state (FIGS. 14 and 15).

In addition to the components of the CAF-1 complex, shRNAs targeting Ube2i, a key enzyme of the SUMO pathway also scored highly in the screening assay. In further studies, shRNAs targeting Sae1 and Uba2a, which are components of the SUMO E1 ligase complex, also validate to strongly enhance iPSC reprogramming, as can be seen in FIG. 13.

Following from this study, the inventors subsequently examined whether transient knockdown of Chaf1a, Chaf1b, or Ube2i during reprogramming affects the ability of the resultant iPSCs to contribute to normal development. To perform this experiment the inventors developed and utilized a dox-inducible shRNAmir expression system. shRNAs and OKSM were simultaneously expressed in reprogrammable MEFs for seven days when Oct4-GFP⁺ cells emerged in CAF-1 and Ube2i depleted cells but not yet in controls. Oct4 GFP⁺ cells were then purified using FACS, followed by dox withdrawal to select for transgene-independent iPSCs. Injection of these iPSCs into blastocysts gave rise to adult high-grade chimeras that efficiently produced germline offspring (Chaff a: 18/20 pups; Chaf1b: 27/27 pups; Ube2i: 17/17 pups) (FIG. 16). These results indicate that silencing of Chaf1a, Chaf1b, or Ube2i during reprogramming does not compromise the potential of iPSCs to differentiate into somatic and germ cell lineages in vivo.

### EXAMPLE 4: FUNCTIONAL INTERACTIONS AND COOPERATIVITY OF CHAF1A, CHAF1B, UBE2I, AND SETDB1 SUPPRESSION DURING REPROGRAMMING

As presented above, the inventors have identified several genes which are important for regulating reprogramming efficiency.

The inventors then decided to examine the effects of combining RNAi-mediated reduction of gene expression of four reprogramming suppressors (Chaf1a, Chaf1b, Ube2i and Setdb1) in a pairwise manner.

Two constitutive miR-E-based shRNAs or control vectors were sequentially transduced in every possible combination and order (64 in total) into reprogrammable MEFs and subsequently induced with dox for seven days. The transgene-independent fraction of Oct4-GFP⁺ cells at day 11 was then measured by flow cytometry and normalized to an empty vector control (FIG. 17). A comparison of these datasets revealed that (i) co-knockdown of different CAF-1 subunits (i.e., shRNAs against Chaf1a/Chaf1a, Chaf1a/Chaf1b and Chaf1b/Chaf1b) slightly reduced the fraction of Oct4-GFP+ cells compared to repression of individual subunits, consistent with the previous observation that strong CAF-1 knockdown compromises cell viability; (ii) co-suppression of Setdb1 and either Chaf1a, Chaf1b or Ube2i reduced overall reprogramming efficiencies compared to suppression of Chaf1a, Chaf1b or Ube2i alone, indicating that knockdown of Setdb1 reduces the enhancing effect of CAF-1 or Ube2i suppression on iPSC generation, possibly due to cellular toxicity; (iii) simultaneous knockdown of either CAF-1 subunit and Ube2i further increased the fraction of Oct4-GFP+ cells in different shRNA combinations, suggesting that these factors may act in independent pathways and/or stages to repress iPSC formation.

### Double Knockdown Methodology

Triple transgenic reprogrammable MEFs were transduced with shRNA expressed from LEPC as previously described and cultured in MEF media. 3 days after retroviral infection cells were sorted for mCherry expression and 40 000 cells were replated per well of a 6 well dish. On the next day those cells were infected with the corresponding second shRNA expressed from LENC. 24 h later cells were cultured in DMEM supplemented with 15% FBS, 100 U ml⁻¹ penicillin, 100 μg ml⁻¹ streptomycin, sodium pyruvate (1 mM), L-glutamine (4 mM), 1000 U/ml LIF, 0.1 mM 2-Mercaptoethanol and 1 μg ml⁻¹, 50 μg ml⁻¹ sodium ascorbate, and doxycycline at 37° C. with lox oxygen (4.5% 02). Starting 36 h after the 2nd shRNA transduction, cell culture media was supplemented with 0.5 μg ml⁻¹ G418 for 3 days and 0.25 μg ml⁻¹ G418 for additional 3 days to ensure double infection. After 7 days of OSKM transgene induction cells were cultured in ESC medium for additional 4 days without doxycycline to withdraw OSKM at 37° C. with 5% CO2. Cells were analyzed for Oct4-GFP expression using a FACS BD LSRFortessa (BD Biosciences).

### EXAMPLE 5: SUPPRESSION OF CAF-1 ACCELERATES iPSC FORMATION

The inventors further decided to examine the effects of the gene expression reduction in the absence of the identified chromatin barriers, by following the emergence of Oct4-GFP⁺ cells over time. While suppression of Ube2i promoted Oct4-GFP activation slightly earlier than controls (day six with Ube2i shRNA vs. day nine with Renilla shRNA), the suppression of either CAF-1 subunit triggered a dramatic acceleration of this process and consistently generated Oct4-GFP+ cells as early as four days of OKSM expression (FIG. 3). Similar effects were observed by flow cytometry-based analysis of Nanog expression (FIG. 15).

In addition to the above, study, the inventors further examined the ability of the shRNA molecules to facilitate transgene-independent clonal growth, a hallmark of authentic iPSCs. Suppression of either CAF-1 subunit or Ube2i indeed gave rise to transgene-independent Oct4-GFP⁺ cells after as little as five days of OSKM expression whereas transgene-independent iPSCs were first detectable by day nine in control shRNA-treated cells (FIG. 4).

The identification of Chaf1a, Chaf1b and Ube2i as clear top hits in a multiplexed chromatin-focused RNAi screen and the validation that suppression of these factors dramatically enhances and (in case of Chaf1a and Chaf1b) accelerates iPSC reprogramming, establishes these factors as as novel major “roadblocks” of iPSC reprogramming. Furthermore, these discoveries establish the suppression of these factors as an novel experimental tool to increase the efficacy of iPSC reprogramming regimens.

### Additional Data

Presented below are mature siRNA guide sequences of shRNAmirs that lead to enhanced iPS reprogramming in the multiplexed RNAi screen (protocol outlined in Examples 1 and 2).

| shRNAName | siRNA Guide | SEQ ID No |
|---|---|---|
| Chaf1a.3118 | UUUACAAUUUGGAAGUAGCUG | SEQ ID NO: 3 |
| Chaf1b.1221 | AAAUGUGCAAUAGCCAUCCGU | SEQ ID NO: 4 |
| Chaf1b.1262 | UCUUUCAAAGGUAUGCCAAGU | SEQ ID NO: 5 |
| Chaf1a.576 | UAUGUGACAAGUGAUGUCUGA | SEQ ID NO: 6 |
| Chaf1a.164 | UGUAUUAACCUCUUCACUGGG | SEQ ID NO: 7 |
| Chaf1b.271 | UAAGAUUGGAGAGAAAUUCCA | SEQ ID NO: 8 |
| Chaf1a.2120 | UUCAAGUCGGAACUCGUGCAG | SEQ ID NO: 9 |
| Ube2i.414 | UUGUUUUGGCAGUAAAUUGUG | SEQ ID NO: 10 |
| Chaf1a.1990 | UUACUUUGUGGUUCUCAGGGU | SEQ ID NO: 11 |
| Setdb1.1142 | UUAUAUUCCUCUAUGAAGUCU | SEQ ID NO: 12 |
| Prdm11.1698 | UAGCCUGCCUCUGUCACCUGA | SEQ ID NO: 13 |
| Smyd5.2042 | UUAUAGAGCACAAUCUGUCAU | SEQ ID NO: 14 |
| Chaf1b.365 | UUCCACAACAGAAUGACGGCA | SEQ ID NO: 15 |
| Brd4.538 | UAAUCUUAUAGUAAUCAGGGA | SEQ ID NO: 16 |
| Ube2i.258 | GAAGGAUACACGUUUGGAUGA | SEQ ID NO: 17 |
| Meaf6.393 | UAAGUCUGGAGAAGUAUCGCU | SEQ ID NO: 18 |
| Chaf1b.899 | UUUCUGGAGAACACAUAAGUG | SEQ ID NO: 19 |
| Dnmt3a.4178 | UUAAUAUUUCUUCAACAGCUA | SEQ ID NO: 20 |
| Kdm4a.1596 | UUCUAGUUUGACAUUCUUCAG | SEQ ID NO: 21 |
| Bcor.5193 | UAUAACACACUGUACACAGUG | SEQ ID NO: 22 |
| Chd1l.2303 | UUCUUGGUUCAGCUGAUCGUA | SEQ ID NO: 23 |
| Zfp740.577 | UUGAGGUGGUAACUGCUCCGA | SEQ ID NO: 24 |
| Zhx3.2912 | UUAUAGUCUUCGUACCACUUG | SEQ ID NO: 25 |
| Ncoa6.4572 | UUUUGUUCUCUUCAACACUGG | SEQ ID NO: 26 |
| Ube2i.2368 | UUAAUUAGAGCAUUUGUAGAU | SEQ ID NO: 27 |
| Pogz.3420 | UUUUCUGCUACAUGCUUCGGU | SEQ ID NO: 28 |
| Suv39h1.1211 | UGUAGUCGCUCAUCAAGGUUG | SEQ ID NO: 29 |
| Trim28.1367 | UUUUUCUGAAGUGUGGCAUGU | SEQ ID NO: 30 |
| Ube2a.534 | UACUAUUGCAGAAACACGCUU | SEQ ID NO: 31 |
| Ube2i.353 | UUAGAAGUUCUUGUAUUCCUA | SEQ ID NO: 32 |
| Yeats4.474 | UAUGAUUUCAAAUUCACCCCA | SEQ ID NO: 33 |
| Chd4.3421 | UACGUUCAUAUUUAUAACCUU | SEQ ID NO: 34 |
| Daxx.400 | UUAAUGUACACAUAGAUCUUA | SEQ ID NO: 35 |
| Setdb1.3828 | UUCAAGUUUGGCAUCAAUGAU | SEQ ID NO: 36 |
| Tfpt.86 | UACUCCUGUCUUGUCGCAGUG | SEQ ID NO: 37 |
| Smarcc1.1051 | UAGGUUUCCUCUUCCUAGAGU | SEQ ID NO: 38 |
| Ubr4.3190 | UUGUUUGAUAAGGUGAUCCUU | SEQ ID NO: 39 |
| Gtf3c5.2350 | UUUUGGAAGAGCUACUUCCUG | SEQ ID NO: 40 |
| Mta3.442 | UACAACACGGAUUCUGUCUCA | SEQ ID NO: 41 |
| Chaf1b.357 | UAGAAUGACGGCAUCAUCUCC | SEQ ID NO: 42 |
| Nono.2431 | UAUUUUAAUAAGGUGAUGCUG | SEQ ID NO: 43 |
| Hdgfl1.1530 | UAUAUGUAUGACUAAAGGCUU | SEQ ID NO: 44 |
| Jmjd6.1631 | UUAUUAAAUAGGUAAAGGGUU | SEQ ID NO: 45 |
| Mbd4.449 | AUUAGCAAGUGAACGUUUUGA | SEQ ID NO: 46 |
| Chaf1a.407 | UCGAUAAUGACCACACUCGGU | SEQ ID NO: 47 |
| L3mbtl4.1918 | UUGCAUUUUCACAUUUUCCGU | SEQ ID NO: 48 |
| Prkaa1.3633 | UUAUUUACAAAUCUAUAACUU | SEQ ID NO: 49 |
| Smarce1.1624 | UUUGUUCGCCACUUGCUCUUC | SEQ ID NO: 50 |
| Esco1.4209 | UUAGUUUACAACUAUUCUGUA | SEQ ID NO: 51 |
| Brd4.2112 | UUUGUUGAUAUCUAGACUUAG | SEQ ID NO: 52 |
| Tcf20.4902 | UUUGCUUAUUGACACUACCGA | SEQ ID NO: 53 |
| Atm.135 | UUGAGUGCUAGACUCAUGGUU | SEQ ID NO: 54 |
| Atxn7l3.695 | AUUGUCAUUGAUGUCAUCGUU | SEQ ID NO: 55 |
| Uimc1.283 | UUCGACUGUUUUGUCUUCGUU | SEQ ID NO: 56 |
| Chaf1a.2741 | UUCAUACAGUCUGUGUCAUCC | SEQ ID NO: 57 |
| Nbn.1299 | UUAUAUUGAAUGUUUCUUGUG | SEQ ID NO: 58 |
| Setd2.1771 | UUUAAAUUUAUCAUUCUUGGA | SEQ ID NO: 59 |
| Parp1.3788 | UUAAUUGAGAACUAUAGCCCU | SEQ ID NO: 60 |
| Parp6.1117 | UUAGAGUCCUGUAUGAUAGGA | SEQ ID NO: 61 |
| Taf9.949 | UUUAACUUGUAGUACAAUGGA | SEQ ID NO: 62 |
| Cbx1.1051 | UAAAUGCUCAAAUAUUACUAU | SEQ ID NO: 63 |
| Chd4.2607 | UUCAAAGGAGAACUCAUUUUC | SEQ ID NO: 64 |
| Gadd45b.964 | UAAAGUCUCAGUCUCCUCUUG | SEQ ID NO: 65 |
| Kdm1b.3857 | UUAUAUUGUACUUUGACAGGG | SEQ ID NO: 66 |
| Men1.1684 | UUAGGAAAGAGAGUGUGUAGU | SEQ ID NO: 67 |
| Ube2b.299 | UAUAUUCUUCAGAAAAUUCUA | SEQ ID NO: 68 |
| Ube2i.2107 | UAUAUUAACCAUAUACAUGUG | SEQ ID NO: 69 |
| Ino80d.4338 | UUACACAUCACUUCACAACUG | SEQ ID NO: 70 |
| Lcor.4051 | UAUUAAUUUCAUCUUUUUCUU | SEQ ID NO: 71 |
| Men1.1690 | UCCGCUUUAGGAAAGAGAGUG | SEQ ID NO: 72 |
| Phf21b.3298 | UUAAAAUAGAUUUGUAUCCUA | SEQ ID NO: 73 |
| Prkdc.6455 | UUCUGUAUUAAUAACAAGCUU | SEQ ID NO: 74 |
| Rag2.1630 | UUCAUUGCAAUAAUACUUGUU | SEQ ID NO: 75 |
| Hira.4296 | UAACUUAUAGGGAUAUCCUGA | SEQ ID NO: 76 |
| Hltf.456 | UUUAAUAGCAUUCUUAUCAUA | SEQ ID NO: 77 |
| Phf20l1.2690 | UAUGUUUAUUCUUUAGUAUUC | SEQ ID NO: 78 |
| Taf5I.1873 | UUAAUGUUCUUGAUUCUCUUG | SEQ ID NO: 79 |
| Usp51.1995 | UUAAUUACUGCAAACAAAGAA | SEQ ID NO: 80 |
| Apobec4.48 | UUUCUGUUUCUAUUAGUUCUU | SEQ ID NO: 81 |
| Atrx.2488 | UUUACCUUUAUGAUUCAUCUG | SEQ ID NO: 82 |
| Atrx.1843 | UUGAUAAUCAGCUGAACUCUG | SEQ ID NO: 83 |
| Cbx4.2571 | UUAAUAUUUACAUUCAAGCAG | SEQ ID NO: 84 |
| Csnk2a1.287 | UUAACAUCUGUGUAAACUCUG | SEQ ID NO: 85 |
| Setd2.1196 | UAUAUCUAUCAUCUCUCUCUG | SEQ ID NO: 86 |
| Usp34.1704 | UUUAUUUCUAUUAAUAAGCUG | SEQ ID NO: 87 |
| Mis18bp1.1586 | UUUCUUUAUCUUCUGCAUCUU | SEQ ID NO: 88 |
| Ppib.229 | UUGUAAAUCAAAGUAUACCUU | SEQ ID NO: 89 |
| Prdm12.2349 | UUCGUAAAUAUCAUCUUAGAU | SEQ ID NO: 90 |
| Setd2.3374 | UUUCAGUUUGAGAACAGCCUU | SEQ ID NO: 91 |
| Setdb1.1958 | UUUCUCAUGGGUCUGAUCCGG | SEQ ID NO: 92 |
| Ubr4.11656 | UAGUGUAAUACAAUGCUCCGU | SEQ ID NO: 93 |
| Csnk2a1.1012 | UAUAGUCAUAUAAAUCUUCUG | SEQ ID NO: 94 |
| Map3k12.3110 | UUUCUUUAUAGCUCUAGGGUA | SEQ ID NO: 95 |
| Chd2.1327 | UUAUCUUGCUGUUCAUCACCU | SEQ ID NO: 96 |
| L3mbtl4.1034 | UUAUCUUUUCUAUAAUCCCGA | SEQ ID NO: 97 |
| Mbd4.1229 | UAAUGUCUCAGAAGUAAAGUG | SEQ ID NO: 98 |
| Scml2.2696 | UUUAUAUACACACAAACUGUA | SEQ ID NO: 99 |
| Smarca5.1137 | UUGCUCUUUAUCUCCUAUCAA | SEQ ID NO: 100 |
| Wiz.3831 | UACAUUGAUAACCAAAAGGUG | SEQ ID NO: 101 |
| Atrx.6142 | UUUACUUUCAUCUAGCUUCAG | SEQ ID NO: 102 |

1. A method of preparing a population of iPS cells comprising reducing the amount and/or activity of one or more components of the CAF1 complex, and/or one or more components of the SUMO pathway, in a population of target cells, and (ii) optionally isolating the iPS cells from the target cell population.

2. The method of claim 1 wherein the step of reducing the amount and/or activity of the CAF1 complex comprises reducing the amount and/or activity of CHAF1A, CHAF1B and/or RBBP4 protein in the target cells.

3. The method claim 1 wherein the step of reducing the amount and/or activity of the SUMO pathway comprises reducing the amount and/or activity of SUMO1, SUMO2, SUMO3, SUMO4, SAE1, UBA2, UBE2I, PIAS1, PIAS2, PIAS3, PIAS4, RANBP2, CBX4, NSMCE2, MUL1, HDAC4, HDAC7, TOPORS, FUS, RASD2, TRAF7, SENP1, SENP2, SENP3, SENP5, SENP6 or SENP7.

4. The method of claim 1 wherein the step reducing the amount and/or activity of one or more components of the CAF1 complex comprises administering to the cells one or more agents that inhibit the expression of CHAF1A, CHAF1B and/or RBBP4.

5. The method of claim 1 wherein the step of reducing the amount and/or activity of the SUMO pathway comprises administering to the cells one or more agents that inhibit the expression of SUMO1, SUMO2, SUMO3, SUMO4, SAE1, UBA2, UBE2I, PIAS1, PIAS2, PIAS3, PIAS4, RANBP2, CBX4, NSMCE2, MUL1, HDAC4, HDAC7, TOPORS, FUS, RASD2, TRAF7, SENP1, SENP2, SENP3, SENP5, SENP6 or SENP7.

6. The method of claim 4 wherein the agent is a siRNA or shRNA molecule.

7. The method of claim 1 wherein the method additional comprises reducing the activity SETDB1 in the target cells.

8. The method of claim 1 wherein the agent is a siRNA or shRNA molecule encoded by a transient expression system in the target cells.

9. The method of claim 8 wherein the target cell is exposed to a transient expression system for between 36 to 120 hours.

10. The method of claim 1 wherein the target cells are somatic mammalian cells, preferably, human cells, non-human primate cells, or mouse cells.

11. The method of claim 10 wherein the somatic mammalian cells are fibroblasts, adult stem cells, Sertoli cells, granulosa cells, neurons, pancreatic islet cells, epidermal cells, epithelial cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells or skeletal muscle cells.

12. A population of iPS cells prepared according to the method of claim 1.

13. A method for preparing a population of differentiated cells, comprising (i) preparing a population of iPS cells according to the method of claim 1, (ii) differentiating the iPS cells using a protocol or factor to form a population of differentiated cells.

14. A cell culture media comprising one or more agents that inhibit the expression of CHAF1A, CHAF1B and/or RBBP4, and/or one or more agents that inhibit the expression of SUMO1, SUMO2, SUMO3, SUMO4, SAE1, UBA2, PIAS1, PIAS3, PIA3, PIA4, RANBP2, CBX4, NSMCE2, MUL1, HDAC4, HDAC7, TOPORS, FUS, RASd2, TRAF7, SENP1, SENP2, SENP3, SENP5, SENP6 and/or SENP7.

15. The method of claim 5 wherein the agent is a siRNA or shRNA molecule.
